(19) United States
(12) Patent Application Publication
(10) Pub. No.: US 2016/0346184 A1
(43) Pub. Date: Dec. 1, 2016

(54) METHOD OF IMPROVING HAIR QUALITY BY IMPROVING SCALP HEALTH

(71) Applicant: The Procter & Gamble Company, Cincinnati, OH (US)

(72) Inventors: James Robert Schwartz, West Chester, OH (US); James Patrick Henry, Mason, OH (US); Kathleen Marie Kerr, Okeana, OH (US); Kenneth Robert Wehmeyer, Cincinnati, OH (US); Lijuan Li, Lebanon, OH (US); Haruko Mizoguchi, Fairfield, OH (US)

(21) Appl. No.: 15/165,418

(22) Filed: May 26, 2016

Related U.S. Application Data
(60) Provisional application No. 62/167,553, filed on May 28, 2015.

(51) Int. Cl. A61K 8/58; A61Q 5/02; A61Q 5/12; A61Q 5/00

(52) U.S. Cl. CPC A61K 8/58; A61Q 5/006; A61Q 5/02; A61Q 5/12; A61Q 5/002; A61K 2800/58

(57) ABSTRACT

A method of improving the health of hair emerging from a scalp comprising a reduction of oxidative stress in the scalp by application of a composition resulting in reduction in oxidative stress in pre-emergent hair as demonstrated by reduced oxidative stress in emergent hair.

### FIELD OF THE INVENTION

The present invention relates to a method for improving the quality of hair by improving the health of the scalp from which it emerged.

### BACKGROUND OF THE INVENTION

Hair quality includes attributes such as surface integrity, shine, softness and retention. Achieving and maintaining desired hair quality is traditionally approached by treating the hair after it emerges from the scalp (i.e., post-emergent hair). Typically, this involves coating the hair surface with cosmetic agents to lubricate fiber-fiber interactions and fill in imperfections to improve shine and feel.

Hair is exposed to tremendous physical and chemical challenges once it has emerged from the scalp and becomes exposed to the environment. For shoulder-length hair, the hair at the tips can exceed three years of age. The cumulative impact of ultraviolet light exposure, heat, combing and brushing mechanical stresses and chemical irritants often results in complete physical compromise of the protective layers of the hair fiber, the hair cuticle (Thibaut et al. “Chronological ageing of human hair keratin fibers” Intl. J. Cosm. Sci. 2010, 32, 422-34).

The formative hair fiber exists for approximately two weeks within the scalp skin prior to emerging from the surface. During this time period, the soft fiber slowly hardens (keratinizes) into the familiar fiber we can then see and feel. While the fiber is maturing, it is in intimate contact with the surrounding scalp skin (physiologically, the scalp/hair unit is called the integument). The surface of the forming hair fiber can be negatively impacted by scalp which is generally “unhealthy.” In a tissue that is generally unhealthy, the self-repair process often involves inflammation, which is a complex physiological reaction that involves tissue destruction and re-building (Schellander, F. and R. Marks, The epidermal response to subepidermal inflammation. Brit. J. Dermatol., 1973. 88: p. 363-367).

The common scalp conditions such as dandruff and seborrheic dermatitis have an inflammatory reaction component. Scalp psoriasis also is an inflammatory condition. The hair growing under certain conditions may be compromised during its maturation either because of the surrounding milieu of molecules negatively impacting the hair surface or by resource depletion due to the reparative needs of the scalp skin. The net impact may be alteration of the hair surface, leaving it compromised and less able to defend against the post-emergent environmental insults or alteration of the anchoring strength of the hair fiber. In a large survey of the French population, those concerned about their scalp condition believe this negatively impacts normal retention of hair (Misery, L., et al., Sensitive scalp: does this condition exist? An epidemiological study. Contact Derm, 2008. 58: p. 234-238).

A measure of the health of a tissue such as skin is the oxidative balance or oxidative stress. There are many sources of potential oxidative damage to the skin, such as metabolic activity of resident microbes, normal human energy metabolism, external sources such as ultraviolet light and pollutants as well as some product exposures, such as bleaches. The result is the formation of a range of small molecules collectively termed reactive oxygen species (ROS) that can be damaging to biomolecules such as lipids and proteins that are critical to proper structure and function of the skin. To protect the skin against these molecules, a range of enzymes (such as superoxide dismutase) are normally present to detoxify ROS. The level and activity of this constituitive anti-oxidant system varies depending on age and local and systemic health. In a healthy state, there is a balance between the pro- and anti-oxidant activities. This is termed low oxidative stress. If either the pro-oxidant forces are unusually large or the anti-oxidant forces unusually low, the balance is no longer achieved, which is considered an unhealthy state with oxidative stress.

The complexity of the oxidative stress physiology results in many potential measures that are indicative of the degree of oxidative stress. The level of enzymes such as myeloperoxidase (MPO) can be indicative of oxidative stress. Another common metric of oxidative stress is to quantify the level of damaged biomolecules such as proteins or lipids. A common measure is the quantitation of oxidatively modified linoleic acid (octadecenedioic acid) to form HODE (hydroxyoctadecenedioic acid) (Yoshida Bio-markers of lipid peroxidation in vivo: Hydroxyoctadecadienoic acid and hydroxycholesterol BioFactors 2006, 27, 195-202). The measure of damaged biomolecules (such as HODE) has the advantage that they can be quantified in both the scalp and hair as measures of the oxidative stress being experienced by both components of the integument. Not only do these parameters enable assessment of the oxidative stress of each component of the integument, doing so under treatment conditions allows determination of a cause-and-effect relationship amongst the various components as well.

### SUMMARY OF THE INVENTION

In an embodiment, the present invention is directed to a method of improving the health of hair emerging from a scalp comprising a reduction of oxidative stress in the scalp by application of a composition resulting in reduction in oxidative stress in pre-emergent hair as demonstrated by reduced oxidative stress in emergent hair.

A method for improving the quality of hair has been discovered whereby the effects are mediated through the condition of the scalp impacting the pre-emergent hair. Improving the scalp health by reducing local oxidative stress in the scalp milieu surrounding the formative hair reduces the resultant oxidative stress to the pre-emergent hair. This enables the hair to form normally within the scalp thereby emerging intact physically with a surface structure more able to withstand the chemical and physical insults representative of normal exposure.

### BRIEF DESCRIPTION OF THE DRAWINGS

FIG. 1 is a graph showing Level of Normalized HODE in Scalps of Unhealthy and Healthy Scalp Populations.

FIG. 2 is a graph showing Level of Normalized HODE in Hair of Unhealthy and Healthy Scalp Populations.

FIG. 3 is a graph showing Impact of Treatment on Scalp HODE Levels.

FIG. 4 is a graph showing Impact of Treatment on Hair HODE Levels.

### DETAILED DESCRIPTION OF THE INVENTION

While the specification concludes with claims which particularly point out and distinctly claim the invention, it is believed the present invention will be better understood from the following description.

The present invention can comprise, consist of, or consist essentially of the essential elements and limitations of the invention described herein, as well any of the additional or optional ingredients, components, or limitations described herein.

All percentages, parts and ratios are based upon the total weight of the compositions of the present invention, unless otherwise specified. All such weights as they pertain to listed ingredients are based on the active level and, therefore; do not include carriers or by-products that may be included in commercially available materials.

The components and/or steps, including those, which may optionally be added, of the various embodiments of the present invention, are described in detail below.

All documents cited are, in relevant part, incorporated herein by reference; the citation of any document is not to be construed as an admission that it is prior art with respect to the present invention.

All ratios are weight ratios unless specifically stated otherwise.

All temperatures are in degrees Celsius, unless specifically stated otherwise.

Except as otherwise noted, all amounts including quantities, percentages, portions, and proportions, are understood to be modified by the word “about”, and amounts are not intended to indicate significant digits.

Except as otherwise noted, the articles “a”, “an”, and “the” mean “one or more”

Herein, “comprising” means that other steps and other ingredients which do not affect the end result can be added. This term encompasses the terms “consisting of” and “consisting essentially of” The compositions and methods/processes of the present invention can comprise, consist of, and consist essentially of the essential elements and limitations of the invention described herein, as well as any of the additional or optional ingredients, components, steps, or limitations described herein.

Herein, “effective” means an amount of a subject active high enough to provide a significant positive modification of the condition to be treated. An effective amount of the subject active will vary with the particular condition being treated, the severity of the condition, the duration of the treatment, the nature of concurrent treatment, and like factors.

The term ‘skin’ means the outer covering of a vertebrate animal, consisting of two layers of cells, a thick inner layer (the dermis) and a thin outer layer (the epidermis). The epidermis is the external, nonvascular layer of the skin. It is made up, from within outward, of five layers of EPITHELIUM: (1) basal layer (stratum basale epidermidis); (2) spinous layer (stratum spinosum epidermidis); (3) granular layer (stratum granulosum epidermidis); (4) clear layer (stratum lucidum epidermidis); and (5) horny layer (stratum corneum epidermidis).

The term “sample” refers to any preparation from skin or epidermis of a subject.

The term “noninvasive” means a procedure that does not require insertion of an instrument or device through the skin or a body orifice for diagnosis or treatment.

The term “adhesive device” means a device used for the removal of the skin's epidermal layer by using an adhesive or an adhesive material on a substrate. For example, skin samples with adhesive tapes such as D-Squame® (polyacrylate ester adhesives; CuDerm; Dallas Tex.), Durapor, Sebutape™ (acrylic polymer films; CuDern; Dallas, Tex.), Tegaderm™, Duct tape (333 Duct Tape, Nashua tape products), Scotch® Tape (3M Scotch 810, St. Paul, Minn.), Diamond™ (The Sellotape Company; Eindhoven, the Netherlands), Sentega™ (polypropylene tape, Sentega Eiketten BV, Utrecht, The Netherlands) may be used. The adhesive may be any of the commonly used pressure-sensitive-type adhesives or those which solidify quickly upon skin content (such as cynaoacylates). The adhesives may be on flexible or solid backings to make sampling easier. A constant pressure device (e.g. Desquame Pressure Instrument, CuDerm; Dallas, Tex.) can be used to apply pressure to the adhesive device during sampling.

Samples from a tissue may be isolated by any number of means well known in the art. Invasive methods for isolating a sample include the use of needles, for example during blood sampling, as well as biopsies of various tissues, blistering techniques and laser poration. Due to the invasive nature of these techniques there is an increased risk of mortality and morbidity. Further, invasive techniques can inadvertently impact the state of the skin, which could lead to inaccurate or false results. Even further, invasive techniques are difficult to execute on a large population. The invasive technique may result in discomfort to the participant and may provide a greater potential for infection or other side effects. The present invention provides a noninvasive method for measuring biomarkers of oxidative stress and oxidative damage from the skin.

The term “objectively” means without bias or prejudice. Alternatively, any expert or self-assessments are inherently “subjective.”

The term “normalization” and/or ‘normalized” means the degree to which a population of dandruff sufferers approach a state of normal population.

The term “standardization” and/or “standardized” means biomarker values expressed relative to the amount of protein measured on the corresponding adhesive or adhesive article in the case of myeloperoxidase. In the case of oxidized lipids the standardization means the value of oxidized lipid is expressed relative to the corresponding non-oxidized parent lipid. A non-limiting example would be ng oxidized lipid/ng parent lipid or pg myeloperoxidase/μg soluble protein.

The term “baseline” means information gathered at the beginning of a study from which variations found in the study are measured.

### Different Domains of Hair Growth:

The human hair fiber originates in the hair follicle approximately 4 mm deep in the scalp skin. The nascent fiber spends approximately two weeks below the scalp surface while it is hardening and maturing, prior to emerging at the scalp surface. Continued growth is approximately 1 cm per month. This allows for the differentiation of specific regions of the hair fiber, relative to the surface of the scalp. The part of the hair fiber existing below the scalp surface is termed “pre-emergent” hair. As the hair just begins to emerge from the scalp surface and for approximately 8 weeks thereafter, the hair is termed “emergent.” Hair that continues to grow past the 8 week period is then considered “post-emergent.”

### Hair Quality/Health

Healthy, high quality hair is desired by all. Many factors can compromise the quality and health of hair, including oxidative stress (Trueb, R. Oxidative Stress in Ageing of Hair Intl J Trichol 2009, 1, 6-14). The consequences of oxidative stress include a rough surface due to insufficient cuticle integrity, decreased shine, compromised anchoring strength and depigmentation. Thus, a relevant measure of hair health and quality is its level of oxidative stress. While there are a number of potential biomarkers of oxidative stress, HODE is widely accepted as a barometer of oxidative stress and, thereby, hair health and quality.

In a further embodiment of the present invention, there is a number of Alternative “Noninvasive” Sampling Methods that may be used.

Sebutape™: This is a noninvasive approach in that Sebutape™ (acrylic polymer film; CuDerm; Dallas, Tex.) is only very mildly adhesive and may be applied to and removed from even visibly inflamed skin without causing discomfort. Biomarkers recovered/assayed by this technique have included proteins (e.g., cytokines), peptides (e.g., neuropeptides), and small molecules (lipids) Historically, this tape is manufactured and sold for sebum collection and can, therefore, be useful for lipid analysis.

D-Squame®: D-Squame® tape is a polyacrylate ester adhesive also manufactured by CuDerm. It may be used to recover the same biomarkers as Sebutape™ but also removes certain epidermal structural proteins (e.g., keratins, involucrin). It has also been used to recover cortisol and serum albumin as systemic inflammatory markers, and small molecules (histamine) and stratum corneum lipids.

Cup Scrubs: Cup scrubs extract proteins directly from the surface of the skin, usually in the presence of buffer, a nonionic surfactant or an organic solvent (ex. ethanol). Cup scrubs are primarily used for recovery of soluble biomarkers such as cytokines, but can also be used to recover small organic molecules. Many more cytokines can be recovered and quantified from cup scrubs than from tape strips. This could be due to several reasons. (a) Due to the presence of detergents and their liquid nature, cup scrubs most likely sample a different protein population than do tape strips. (b) With cup scrubs, cytokines do not have to be further extracted after sample collection since they already are in solution.

Hair plucks: Plucking hairs is the process of removing human or animal hair by mechanically pulling the item from the owner's body usually with tweezers. The follicular region of the hair pluck is extracted usually in the presence of buffer and a nonionic surfactant for recovery of soluble protein biomarkers such as cytokines, and can also be extracted with an organic solvent to recover small organic molecules like lipids.

Animal (i.e. Dog) Collection Method: D-Squame®: D-Squame™ tape samples are collected on dogs' skin via parting their fur (without shaving). A variety of biomarkers related to skin inflammation, differentiation and barrier integrity can be analyzed from the tapes including total protein, soluble protein, skin multiple analyte profile (skin MAP), skin cytokines and stratum corneum lipids (ceramides, cholesterol, fatty acids).

In an embodiment of the present invention, the present invention provides a method and analysis for noninvasively obtaining a sample for use in isolating myeloperoxidase and oxidized lipids.

In an embodiment, the use of an adhesive device can be used to achieve such sampling. In preparation for such a sampling study for a dandruff sampling, at a baseline visit, a qualified screening grader will complete adherent scalp flaking score (ASFS) grading for each subject and the highest flaking octant will be identified for tape strip sampling. The highest flaking octant will be sampled at baseline and various time points. Tape strips samples will be collected from each subject at each time point.

The tape strip sampling is repeated additional times, as needed, at the same site placing each D-Squame® tape disc on top of the prior sampled area. The D-Squame® tapes after sample collection are placed into the appropriately labeled wells in a labeled plate.

Following the sampling, an extraction and quantitation procedure is conducted. In an embodiment of the present invention, quantitation of myeloperoxidase and oxidized lipids from extracts of D-Squame® Tape Samples can be conducted via analysis by either antibody-based immunoassay or by LC/MS/MS. In this embodiment of the present invention, the sample extraction in preparation for antibody based analysis or LC/MS/MS analysis is performed.

For the Myeloperoxidase method, appropriate standard extraction buffers are added to each collection tube and then extracted on ice using sonication for 30 min. Each extract solution is isolated from the tape strip and an aliquot of each sample is placed into a specified position of a 96-well polypropylene plate. Aliquots of the extracts of D-Squame® Tape samples are then supplemented with conventional reagents, such as albumin, to help prevent loss of analytes to the walls of labware, transferred into 96-well polypropylene deep well plates and frozen at −80° C. for myeloperoxidase analysis. A separate aliquot is not supplemented with reagents and is analyzed for soluble protein using a BCA™ Protein Assay Kit, Pierce catalog #23227.

Following the extraction process, Myeloperoxidase standards and controls can be prepared by conventional methods. Myeloperoxidase will be quantitated with a myeloperoxidase immunoassay kit from Mesoscale Discovery. The result can be reported as the amount of Myeloperoxidase/tape strip or the result can be standardized by dividing by the amount of myeloperoxidase by the amount of the protein that is also found in the tape strip extract. The protein method has been described separately. Data analysis is conducted by standard statistical methods and calculations.

In a further embodiment of the present invention, quantitation of oxidized lipids from extracts of the adhesive article, tape strips, can be conducted using gradient reversed-phase high performance liquid chromatography with tandem mass spectrometry (HPLC/MS/MS).

Tape strips (single or multiple tape strips) obtained from the scalp of human subjects are placed into individual polypropylene amber vials or glass amber vials, and then extracted with extraction solvent (methanol with 0.1% butylated hydroxytoluene, w/v) using vortexing for 10 min. The standards and the extracts of the scalp tape strips are analyzed using gradient reversed-phase high performance liquid chromatography with tandem mass spectrometry (HPLC/MS/MS). Analytes (oxidized or non-oxidized lipids) listed in Table 1 and the ISTDs are monitored by positive ion electrospray (ESI). A standard curve is constructed by plotting the signal, defined here as the peak area ratio (peak area analyte/peak area ISTD) or peak area analyte only, for each standard versus the mass of each analyte for the corresponding standard. The mass of each analyte in the calibration standards and human scalp extract samples are then back-calculated using the generated regression equation. The result can be reported as the mass of oxidized lipid/tape strip or the result can be standardized by dividing by the amount of oxidized lipid by the amount of the corresponding parent non-oxidized lipid that is also found in the tape strip extract. Additionally results could be reported by standardizing the amount of oxidized lipid by the amount of corresponding protein found in the tape strip extract. Standardization could also be done by collecting the cells removed, drying them and weighing them.

TABLE 1
Analytes

9/13-HODE
(±)-9-hydroxy-10E, 12Z-octadecadienoic acid and (±)-13-hydroxy-10E,
12Z-octadecadienoic acid (HODE)
9/13-HpODE
(±)-9-hydroperoxy-10E, 12Z-octadecadienoic acid and (±)-13-
hydroperoxy-10E, 12Z-octadecadienoic acid (HODE)
CH-OOH
Cholesterol Hydroperoxide
SQ-OOH
Squalene Hydroperoxide
Oxidosqualene
5α,6α-epoxy-Chol
5α,6α-epoxy-cholesterol
4β-OH-Chol
4β-hydroxycholesterol
7β-OH-Chol
7β-hydroxycholesterol
Linoleic acid
Cholesterol
Squalene

Methodology Extension

Although the exact procedure used is described above, there are a number of alternate approaches that could be taken for a number of the steps outlined above that are logical extensions. The extraction solvents employed for isolating Myeloperoxidase and oxidized lipids from the tape strip can be any appropriate aqueous, organic or organic/aqueous mixture that provides a suitable recovery. LC/MS/MS and antibody-based immunoassays are generally recognized as the state-of-the-art approaches for the quantitative analysis of organic molecules in biological matrices due to their high selectivity and sensitivity. However, any analytical technique and or other approach providing the required sensitivity and selectivity could be employed. For example, other methods for assessing biomolecules have been employed including: capillary electrophoresis, supercritical fluid and other chromatographic techniques and/or combinations thereof. Similarly, instrumental approaches without separation techniques have also been employed including nuclear magnetic resonance spectroscopy, mass spectrometry, electrochemical and fluorometric assays. Additionally, ligand binding approaches such competitive and non-competitive enzyme linked immunosorbent assays (ELISAs) and radioimmunoassay (RIA) or other labeling schemes have also been employed. Enzyme-based assays have a long history of use in the analysis of proteins. Bioassay using either cell-based or tissue-based approaches could have also been used as the means of detection. In an embodiment of the present invention, quantitation of biomarkers of oxidative stress and oxidative damage from hair plucks can be carried out with the same basic extraction and analysis methods as used for tape strip samples.

### Protein Determination of Tape Strip Extracts:

The level of myeloperoxidase on tape strip samples of skin measured using a suitable methodology described above can be standardized using amount of protein found in the tape strip extract. Standardization is done by dividing the amount of myeloperoxidase by the amount of protein in the tape strip extract.

The amount of protein in the tape strip extract or an equivalent matrix that is used to determine the Myeloperoxidase level on skin can be determined using variety of protein determination methods described in the literature. Examples of such methods include total nitrogen determination, total amino acid determination and protein determination based on any colorimetric, flurometric, luminometric methods. These methods may or may not involve further sample preparation of the tape strip extract prior to protein determination. A non-limiting example of a specific method for protein determination in the tape strip extract is given below. A comprehensive review of protein determination methods, their applicability and limitations are described in the Thermo Scientific Pierce Protein Assay Technical Handbook that can be downloaded from the following link, incorporated by reference herein. www.piercenet.com/Files/1601669_PAssayFINAL_Intl.pdf. Further information related to protein determination can be found at Redinbaugh, M. G. and Turley, R. B. (1986). Adaptation of the bicinchoninic acid protein assay for use with microtiter plates and sucrose gradient fractions. Anal. Biochem. 153, 267-271, incorporated by reference herein.

Adhesive tapes sampled from human skin will be extracted and analyzed for protein content using the BCA™ Protein Assay Kit (Pierce). The tape strips sampled from human skin will be extracted with a conventional extraction buffer. Following extraction, aliquots of the tape extracts will be transferred into 96-well polypropylene deep well plates and stored at 2-8° C. for protein determination.

The BCA™ Protein Assay Kit is based on the reduction of Cu²⁺ to Cu¹⁺ by proteins in an alkaline medium coupled with the sensitive and selective colorimetric detection of Cu⁺¹ by bicinchoninic acid (BCA). The purple-colored reaction product, formed by chelation of 2 molecules of BCA with one Cu¹⁺ ion, exhibits strong absorbance at a wavelength of 562 nm. The optical density (OD) is measured using a microplate reader. Increasing concentrations of Bovine Serum Albumin (BSA), expressed in micrograms per milliliter (μg/mL), are used to generate a calibration curve in the assay. Appropriate assay QC's prepared from the BSA stock solution will be used to monitor assay performance during sample analysis.

In an alternative embodiment of the present invention, protein determination can be done direct measurement of protein on an adhesive or an adhesive article such as protein measurement with a SquameScan® 850A (CuDerm Corporation, Dallas, Tex.).

In a further embodiment of the present invention, additional oxidative stress markers (in addition to unsaturated fatty acid hydroperoxides/hydroxides, cholesterol hydroperoxides/hydroxides and squalene hydroperoxide/oxide/hydroxides) may include the following:

### Heat Shock Protein (Hsp) 27

The cytoprotective properties of Hsp27 result from its ability to modulate reactive oxygen species and to raise glutathione levels.

Heat shock protein 27 (HSP27) belongs to the small molecular weight heat shock protein (HSP) family (12-43 kDa). HSP27 and other members of the small HSP family share a conserved c-terminal domain, the α-crystallin domain, which is identical to the vertebrate eye lens α-crystallin [1]. HSP27 is initially characterized in response to heat shock as a protein chaperone that facilitates the proper refolding of damaged proteins. Continued investigation of HSP27 revealed that the protein responds to cellular stress conditions other than heat shock; for example oxidative stress and chemical stress. During oxidative stress, HSP27 functions as an antioxidant, lowering the levels of reactive oxygen species (ROS) by raising levels of intracellular glutathione and lowering the levels of intracellular iron.

### Oxidative Modification of Proteins

The oxidation of proteins in biological systems occurs by spontaneous autoxidation of cysteinyl thiols, interactions of proteins with reactive oxygen species (ROS) and by deliberate and controlled reactions catalyzed by oxidases. Reaction of proteins with ROS can result in oxidation of cysteine, methionine, tyrosine, phenylalanine and tryptophan residues. Methione oxidation is monitored by determining methionine sulfoxide, oxidation of tyrosine can by the amount of dityrosine formed, oxidation of phenylalaninine by the formation of o-tyrosine and m-tyrosine, oxidation of tryptophan residues is followed by monitoring N-formylknurenine, kynurenine and/or quinolinic acid. Also, the covalent and oxidative modification of albumin cys34 residue has been suggested as a specific biomarker of mild oxidative stress. Usually, these protein modification adduct residues are determined after exhaustive enzymatic hydrolysis or chemical digestion.

Proteins can be modified via oxidative pathways involving the formation of protein carbonyl groups mainly formed from lysine, proline and arginine residues. Lysine forms 2-aminoadipic semialdehyde (AASA) via oxidative deamination and glutamic semialdehyde (GSA) is formed by oxidation of proline and arginine residues. The AASA and GSA can be detected after reduction to give 6-hydroxy-2-aminocaproic acid and 5-hydroxy-2-aminovaleric acid, respectively. Protein carbonylation can also be determined by ELISA based approaches following derivitization with 2,4-dintirophenylhydrazine.

Additionally, proteins can be modified due to oxidative pathways via reaction with α,β-unsaturated alkenals formed from the oxidation of polyunsaturated fatty acids (see Reaction Products of -unsaturated alkenals with Protein and Mercapturic Acid Pathway) and by the formation of early glycation adducts (EGA) and advanced glycation products (AGEs) with sugars (see EGA and AGEs).

### DNA Oxidation and Hydroxylated Nucleotides¹

DNA damage is generally one measure of oxidative stress with the main cause due to free radical damage caused by endogenous reactive oxygen species (ROS) Oxidative damage to intact DNA can be measured using the COMET assay. Oxidative damage can be measured by monitoring a variety of hydroxylated nucleotides including 8-hydroxydexoygaunosine (8OHdG) which is also referred to as 8-oxodeoxyguanosine (8-oxodG), 4,6-diamino-5-formamidopyrimidine (FapyAde) and 2,6-diamino-4-hydroxy-5-formamidopyrimidine (FapyGua). (¹) Biomarkers for Antioxidant Defense and Oxidative Damage: Principles and Practical Applications; G. Aldini, K-J Yeum, E. Niki and R. M. Russell, eds, Wiley-Blackwell, Iowa, 2010, incorporated herein by reference.

### Isoprostanes¹

Isoprostanes are a series of prostaglandin-like isomers formed from the free-radical catalyzed oxidation of the polyunsaturated fatty acids ((PUFAs) such as arachidonic acid (AA) and the omega-3 eicosapentaenoic acid (EPA), typically the oxidation of PUFAs occurs to the phospholipid form. Isoprostanes derived from AA oxidation result in an F-type prostane rings (referred to as F₂-Isoprostanes) and give rise to the 5-F₂-series IsoP, 8-F₂-series IsoP, 12-F₂-series IsoP and 15-F₂-series IsoPs8-series. Similarly, D/E-ring and A/J-ring isoprostanes are also formed are formed from AA also. Isoprosanes of the F₃-family are similarly formed from EPA and give rise to 5-F₃-series, 8-F₃-series, 11-F₃-series, 12-F₃-series, 15-F₃-series and 18-F₃-series Isoprostanes.

### α,β-Unsaturated Aldehydes¹

Oxidation of polyunsaturated fatty acids often occurs in response to oxidative stress resulting in a radical driven formation of fatty acid hydroperoxides which can undergo further reaction to give a wide diversity of α,β-unsaturated alkenals in biological systems, such as malondialdehyde, acrolein, crotonaldehyde, 4-hydroxynonenal, 4-hydroxyhexenal and 4-oxo-nonenal. These α,β-unsaturated alkenals have been followed as biomarkers of oxidative damage.

Reaction Products of α,β-Unsaturated Alkenals with Protein and Mercapturic Add Pathway

The α,β-unsaturated alkenals are reactive electrophiles and form products with a number of nucleophilic compounds including covalent protein adducts referred to as advanced lipoxidation end-products (ALE) such as hexanoyl-lysine, hexanoyl-histidine, Ne-(3-methylpyridinium)lysine and with mercapturic acid compounds (glutathione, cysteine and mercapturic acid) to form thioethers (ex. 1,4-dihydronone mercapturic acid, 3-hydroxypropylmercapturic acid and carboxyethylmercapturic acid).

### Early Glycation Adducts (EGA) and Advanced Glycation End Products (AGE)

Glycation of proteins is a non-enzymatic complex series of parallel and sequential reactions collectively called the Maillard reaction and occurs in all tissues and body fluids. Glycation adducts can be formed by the reaction of proteins with glucose and reactive α-oxoaldehydes such as glyoxal, methylglyoxal and 3-deoxyglucosone and other saccharide derivatives. Early stage reactions in glycation lead to the formation of fructosyl-lysine and N-terminal amino acid residue-derived fructosamines and are referred to as Early Glycation Adducts (EGA). Later stage reactions form stable end stage adducts called advanced glycation end products such as monolysyl adducts (carboxymethyl lysine (CML), carboxyethyl lysine (CEL) and pyrraline), monovalyl adducts (carboxymethyl valine (CMV) and carboxyethylvaline (CEV)), hydroimidazolones, bis(lysyl) imidazolium crosslinks (GOLD, MOLD, DOLD) and pentosidine derived from a cross link of lysine and arginine. The EGAs and AGEs are released when proteins are degraded by proteolysis or when proteins are degraded by chemical lysis. The formation and accumulation of AGEs have been implicated in the progression of age-related diseases. Research over the last 20 years has implicated AGEs in most of the diseases associated with aging. CML may be a general marker of oxidative stress and long term damage to protein in aging, atherosclerosis, and diabetes.

### Antioxidants as Biomarkers of Oxidative Stress¹

Endogenous antioxidants play a key defense role in controlling oxidative damage caused by radical's mechanisms. Key endogenous antioxidants include ascorbic acid (AsA), glutathione (GSH), α-tocopherol and Coenzyme Q 10 (CoQ). Changes in the levels of these endogenous redox (oxidized and reduced forms) antioxidants can be used as a measure of oxidative stress.

Scalp Active Material

In an embodiment of the present invention, the composition comprises a scalp active material, which may be an anti-dandruff active material. In an embodiment, the anti-dandruff active is selected from the group consisting of: pyridinethione salts; zinc carbonate; azoles, such as ketoconazole, econazole, and elubiol; selenium sulphide; particulate sulfur; keratolytic agents such as salicylic acid; and mixtures thereof. In an embodiment, the anti-dandruff particulate is a pyridinethione salt. Such anti-dandruff particulate should be physically and chemically compatible with the components of the composition, and should not otherwise unduly impair product stability, aesthetics or performance.

Pyridinethione particulates are suitable particulate anti-dandruff actives for use in composition of the present invention. In an embodiment, the anti-dandruff active is a 1-hydroxy-2-pyridinethione salt and is in particulate form. In an embodiment, the concentration of pyridinethione anti-dandruff particulate ranges from about 0.01% to about 5%, by weight of the composition, or from about 0.1% to about 3%, or from about 0.1% to about 2%. In an embodiment, the pyridinethione salts are those formed from heavy metals such as zinc, tin, cadmium, magnesium, aluminium and zirconium, generally zinc, typically the zinc salt of 1-hydroxy-2-pyridinethione (known as “zinc pyridinethione” or “ZPT”; zinc pyrithione), commonly 1-hydroxy-2-pyridinethione salts in platelet particle form. In an embodiment, the 1-hydroxy-2-pyridinethione salts in platelet particle form have an average particle size of up to about 20 microns, or up to about 5 microns, or up to about 2.5 microns. Salts formed from other cations, such as sodium, may also be suitable. Pyridinethione anti-dandruff actives are described, for example, in U.S. Pat. No. 2,809,971; U.S. Pat. No. 3,236,733; U.S. Pat. No. 3,753,196; U.S. Pat. No. 3,761,418; U.S. Pat. No. 4,345,080; U.S. Pat. No. 4,323,683; U.S. Pat. No. 4,379,753; and U.S. Pat. No. 4,470,982.

In an embodiment, in addition to the anti-dandruff active selected from polyvalent metal salts of pyrithione, the composition further comprises one or more anti-fungal and/or anti-microbial actives. In an embodiment, the anti-microbial active is selected from the group consisting of: coal tar, sulfur, charcoal, whitfield's ointment, castellani's paint, aluminum chloride, gentian violet, octopirox (piroctone olamine), ciclopirox olamine, rilopirox, MEA-Hydroxyoctyloxypyridinone; strobilurins such as azoxystrobin and metal chelators such as 1,10-phenanthroline, undecylenic acid and its metal salts, potassium permanganate, selenium sulphide, sodium thiosulfate, propylene glycol, oil of bitter orange, urea preparations, griseofulvin, 8-hydroxyquinoline ciloquinol, thiobendazole, thiocarbamates, haloprogin, polyenes, hydroxypyridone, morpholine, benzylamine, allylamines (such as terbinafine), tea tree oil, clove leaf oil, coriander, palmarosa, berberine, thyme red, cinnamon oil, cinnamic aldehyde, citronellic acid, hinokitol, ichthyol pale, Sensiva SC-50, Elestab HP-100, azelaic acid, lyticase, iodopropynyl butylcarbamate (IPBC), isothiazalinones such as octyl isothiazalinone, and azoles, and mixtures thereof. In an embodiment, the anti-microbial is selected from the group consisting of: itraconazole, ketoconazole, selenium sulphide, coal tar, and mixtures thereof.

In an embodiment, the azole anti-microbials is an imidazole selected from the group consisting of: benzimidazole, benzothiazole, bifonazole, butaconazole nitrate, climbazole, clotrimazole, croconazole, eberconazole, econazole, elubiol, fenticonazole, fluconazole, flutimazole, isoconazole, ketoconazole, lanoconazole, metronidazole, miconazole, neticonazole, omoconazole, oxiconazole nitrate, sertaconazole, sulconazole nitrate, tioconazole, thiazole, and mixtures thereof, or the azole anti-microbials is a triazole selected from the group consisting of: terconazole, itraconazole, and mixtures thereof. When present in the composition, the azole anti-microbial active is included in an amount of from about 0.01% to about 5%, or from about 0.1% to about 3%, or from about 0.3% to about 2%, by total weight of the composition. In an embodiment, the azole anti-microbial active is ketoconazole. In an embodiment, the sole anti-microbial active is ketoconazole.

The present invention may also comprise a combination of anti-microbial actives. In an embodiment, the combination of anti-microbial active is selected from the group of combinations consisting of: octopirox and zinc pyrithione, pine tar and sulfur, salicylic acid and zinc pyrithione, salicylic acid and elubiol, zinc pyrithione and elubiol, zinc pyrithione and climbasole, octopirox and climbasole, salicylic acid and octopirox, and mixtures thereof.

In an embodiment, the composition comprises an effective amount of a zinc-containing layered material. In an embodiment, the composition comprises from about 0.001% to about 10%, or from about 0.01% to about 7%, or from about 0.1% to about 5% of a zinc-containing layered material, by total weight of the composition.

Zinc-containing layered materials may be those with crystal growth primarily occurring in two dimensions. It is conventional to describe layer structures as not only those in which all the atoms are incorporated in well-defined layers, but also those in which there are ions or molecules between the layers, called gallery ions (A. F. Wells “Structural Inorganic Chemistry” Clarendon Press, 1975). Zinc-containing layered materials (ZLMs) may have zinc incorporated in the layers and/or be components of the gallery ions. The following classes of ZLMs represent relatively common examples of the general category and are not intended to be limiting as to the broader scope of materials which fit this definition.

Many ZLMs occur naturally as minerals. In an embodiment, the ZLM is selected from the group consisting of: hydrozincite (zinc carbonate hydroxide), basic zinc carbonate, aurichalcite (zinc copper carbonate hydroxide), rosasite (copper zinc carbonate hydroxide), and mixtures thereof. Related minerals that are zinc-containing may also be included in the composition. Natural ZLMs can also occur wherein anionic layer species such as clay-type minerals (e.g., phyllosilicates) contain ion-exchanged zinc gallery ions. All of these natural materials can also be obtained synthetically or formed in situ in a composition or during a production process.

Another common class of ZLMs, which are often, but not always, synthetic, is layered double hydroxides. In an embodiment, the ZLM is a layered double hydroxide conforming to the formula [M²⁺₁₋ₓM³⁺ₓ(OH)₂]^{x+}A^{m-}_{x/m}.nH₂O wherein some or all of the divalent ions (M²⁺) are zinc ions (Crepaldi, E L, Pava, P C, Tronto, J, Valim, J B J. Colloid Interfac. Sci. 2002, 248, 429-42).

Yet another class of ZLMs can be prepared called hydroxy double salts (Morioka, H., Tagaya, H., Karasu, M, Kadokawa, J, Chiba, K Inorg. Chem. 1999, 38, 4211-6). In an embodiment, the ZLM is a hydroxy double salt conforming to the formula [M²⁺₁₋ₓM²⁺₁₊ₓ(OH)_{3(1-y)}]⁺Aⁿ⁻_{(1=3y)/n}.nH₂O where the two metal ions (M²⁺) may be the same or different. If they are the same and represented by zinc, the formula simplifies to [Zn₁₊ₓ(OH)₂]^{2x+}2xA⁻.nH₂O. This latter formula represents (where x=0.4) materials such as zinc hydroxychloride and zinc hydroxynitrate. In an embodiment, the ZLM is zinc hydroxychloride and/or zinc hydroxynitrate. These are related to hydrozincite as well wherein a divalent anion replace the monovalent anion. These materials can also be formed in situ in a composition or in or during a production process.

In an embodiment, the composition comprises basic zinc carbonate. Commercially available sources of basic zinc carbonate include Zinc Carbonate Basic (Cater Chemicals: Bensenville, Ill., USA), Zinc Carbonate (Shepherd Chemicals: Norwood, Ohio, USA), Zinc Carbonate (CPS Union Corp.: New York, N.Y., USA), Zinc Carbonate (Elementis Pigments: Durham, UK), and Zinc Carbonate AC (Bruggemann Chemical: Newtown Square, Pa., USA). Basic zinc carbonate, which also may be referred to commercially as “Zinc Carbonate” or “Zinc Carbonate Basic” or “Zinc Hydroxy Carbonate”, is a synthetic version consisting of materials similar to naturally occurring hydrozincite. The idealized stoichiometry is represented by Zn₅(OH)₆(CO₃)₂ but the actual stoichiometric ratios can vary slightly and other impurities may be incorporated in the crystal lattice.

In embodiments having a zinc-containing layered material and a pyrithione or polyvalent metal salt of pyrithione, the ratio of zinc-containing layered material to pyrithione or a polyvalent metal salt of pyrithione is from about 5:100 to about 10:1, or from about 2:10 to about 5:1, or from about 1:2 to about 3:1.

### Scalp Health Actives

In an embodiment of the present invention, a scalp health active may be added to further provide scalp benefits. This group of materials is varied and provides a wide range of benefits including moisturization, barrier improvement, anti-fungal, anti-microbial and anti-oxidant, anti-itch, and sensates. Such skin health actives include are not limited to: vitamin E and F, salicylic acid, glycols, glycolic acid, PCA, PEGs, erythritol, glycerin, triclosan, lactates, niacinamide, caffeine, hyaluronates, allantoin and other ureas, betaines, sorbitol, glutamates, xylitols, menthol, menthyl lactate, iso cyclomone, benzyl alcohol, and natural extracts/oils including peppermint, spearmint, argan, jojoba and aloe, sensates, chelants, enzymes, attractants and mixtures thereof.

### Scalp Care Composition

In an embodiment of the present invention, the scalp care composition may be a shampoo composition, a conditioner composition, a leave on composition, or any other conventional scalp care composition. The compositions of the present invention can be in the form of rinse-off products or leave-on products, and can be formulated in a wide variety of product forms, including but not limited to creams, gels, emulsions, foams, mousses and sprays.

Shampoo Composition

Detersive Surfactant

In an embodiment of the present invention, the scalp care composition may be a shampoo composition comprising one or more detersive surfactants, which provides cleaning performance to the composition. The one or more detersive surfactants in turn may comprise an anionic surfactant, amphoteric or zwitterionic surfactants, or mixtures thereof. Various examples and descriptions of detersive surfactants are set forth in U.S. Pat. No. 6,649,155; U.S. Patent Application Publication No. 2008/0317698; and U.S. Patent Application Publication No. 2008/0206355, which are incorporated herein by reference in their entirety.

The concentration of the detersive surfactant component in the shampoo composition should be sufficient to provide the desired cleaning and lather performance, and generally ranges from about 2 wt % to about 50 wt %, from about 5 wt % to about 30 wt %, from about 8 wt % to about 25 wt %, from about 10 wt % to about 20 wt %, about 5 wt %, about 10 wt %, about 12 wt %, about 15 wt %, about 17 wt %, about 18 wt %, or about 20 wt %.

Anionic surfactants suitable for use in the compositions are the alkyl and alkyl ether sulfates. Other suitable anionic surfactants are the water-soluble salts of organic, sulfuric acid reaction products. Still other suitable anionic surfactants are the reaction products of fatty acids esterified with isethionic acid and neutralized with sodium hydroxide. Other similar anionic surfactants are described in U.S. Pat. Nos. 2,486,921; 2,486,922; and 2,396,278, which are incorporated herein by reference in their entirety.

Exemplary anionic surfactants for use in the shampoo composition include ammonium lauryl sulfate, ammonium laureth sulfate, triethylamine lauryl sulfate, triethylamine laureth sulfate, triethanolamine lauryl sulfate, triethanolamine laureth sulfate, monoethanolamine lauryl sulfate, monoethanolamine laureth sulfate, diethanolamine lauryl sulfate, diethanolamine laureth sulfate, lauric monoglyceride sodium sulfate, sodium lauryl sulfate, sodium laureth sulfate, potassium lauryl sulfate, potassium laureth sulfate, sodium lauryl sarcosinate, sodium lauroyl sarcosinate, lauryl sarcosine, cocoyl sarcosine, ammonium cocoyl sulfate, ammonium lauroyl sulfate, sodium cocoyl sulfate, sodium lauroyl sulfate, potassium cocoyl sulfate, potassium lauryl sulfate, triethanolamine lauryl sulfate, triethanolamine lauryl sulfate, monoethanolamine cocoyl sulfate, monoethanolamine lauryl sulfate, sodium tridecyl benzene sulfonate, sodium dodecyl benzene sulfonate, sodium cocoyl isethionate and combinations thereof. In a further embodiment, the anionic surfactant is sodium lauryl sulfate or sodium laureth sulfate.

Suitable amphoteric or zwitterionic surfactants for use in the shampoo composition herein include those which are known for use in shampoo or other personal care cleansing. Concentrations of such amphoteric surfactants range from about 0.5 wt % to about 20 wt %, and from about 1 wt % to about 10 wt %. Non limiting examples of suitable zwitterionic or amphoteric surfactants are described in U.S. Pat. Nos. 5,104,646 and 5,106,609, which are incorporated herein by reference in their entirety.

Amphoteric detersive surfactants suitable for use in the shampoo composition include those surfactants broadly described as derivatives of aliphatic secondary and tertiary amines in which the aliphatic radical can be straight or branched chain and wherein one of the aliphatic substituents contains from about 8 to about 18 carbon atoms and one contains an anionic group such as carboxy, sulfonate, sulfate, phosphate, or phosphonate. Exemplary amphoteric detersive surfactants for use in the present shampoo composition include cocoamphoacetate, cocoamphodiacetate, lauroamphoacetate, lauroamphodiacetate, and mixtures thereof.

Zwitterionic detersive surfactants suitable for use in the shampoo composition include those surfactants broadly described as derivatives of aliphatic quaternaryammonium, phosphonium, and sulfonium compounds, in which the aliphatic radicals can be straight or branched chain, and wherein one of the aliphatic substituents contains from about 8 to about 18 carbon atoms and one contains an anionic group such as carboxy, sulfonate, sulfate, phosphate or phosphonate. In another embodiment, zwitterionics such as betaines are selected.

Non limiting examples of other anionic, zwitterionic, amphoteric or optional additional surfactants suitable for use in the shampoo composition are described in McCutcheon's, Emulsifiers and Detergents, 1989 Annual, published by M. C. Publishing Co., and U.S. Pat. Nos. 3,929,678, 2,658,072; 2,438,091; 2,528,378, which are incorporated herein by reference in their entirety.

The shampoo composition may also comprise a shampoo gel matrix, an aqueous carrier, and other additional ingredients described herein.

Shampoo Gel Matrix

The shampoo composition described herein may comprise a shampoo gel matrix. The shampoo gel matrix comprises (i) from about 0.1% to about 20% of one or more fatty alcohols, alternative from about 0.5% to about 14%, alternatively from about 1% to about 10%, alternatively from about 6% to about 8%, by weight of the shampoo gel matrix; (ii) from about 0.1% to about 10% of one or more shampoo gel matrix surfactants, by weight of the shampoo gel matrix; and (iii) from about 20% to about 95% of an aqueous carrier, alternatively from about 60% to about 85% by weight of the shampoo gel matrix.

The fatty alcohols useful herein are those having from about 10 to about 40 carbon atoms, from about 12 to about 22 carbon atoms, from about 16 to about 22 carbon atoms, or about 16 to about 18 carbon atoms. These fatty alcohols can be straight or branched chain alcohols and can be saturated or unsaturated. Nonlimiting examples of fatty alcohols include, cetyl alcohol, stearyl alcohol, behenyl alcohol, and mixtures thereof. Mixtures of cetyl and stearyl alcohol in a ratio of from about 20:80 to about 80:20 are suitable.

The shampoo gel matrix surfactants may be any of the detersive surfactants described in the detersive surfactants section herein.

The aqueous carrier may comprise water, or a miscible mixture of water and organic solvent, and in one aspect may comprise water with minimal or no significant concentrations of organic solvent, except as otherwise incidentally incorporated into the composition as minor ingredients of other components.

The aqueous carrier useful herein includes water and water solutions of lower alkyl alcohols and polyhydric alcohols. The lower alkyl alcohols useful herein are monohydric alcohols having 1 to 6 carbons, in one aspect, ethanol and isopropanol. Exemplary polyhydric alcohols useful herein include propylene glycol, hexylene glycol, glycerin, and propane diol.

Conditioner Composition

In an embodiment of the present invention, the scalp care composition may be a conditioner composition. The conditioner composition described herein comprises (i) from about 0.025% to about 0.25%, alternatively from about 0.05% to about 0.2%, alternatively from about 0.1% to about 0.15% histidine, by weight of the conditioner composition, and (ii) a conditioner gel matrix. After applying to the hair a conditioner composition as described herein, the method then comprises rinsing the conditioner composition from the hair. The conditioner composition also comprises a conditioner gel matrix comprising (1) one or more high melting point fatty compounds, (2) a cationic surfactant system, and (3) a second aqueous carrier.

### Cationic Surfactant System

The conditioner gel matrix of the conditioner composition includes a cationic surfactant system. The cationic surfactant system can be one cationic surfactant or a mixture of two or more cationic surfactants. The cationic surfactant system can be selected from: mono-long alkyl quaternized ammonium salt; a combination of mono-long alkyl quaternized ammonium salt and di-long alkyl quaternized ammonium salt; mono-long alkyl amidoamine salt; a combination of mono-long alkyl amidoamine salt and di-long alkyl quaternized ammonium salt, a combination of mono-long alkyl amidoamine salt and mono-long alkyl quaternized ammonium salt.

The cationic surfactant system can be included in the composition at a level by weight of from about 0.1% to about 10%, from about 0.5% to about 8%, from about 0.8% to about 5%, and from about 1.0% to about 4%.

### Mono-Long Alkyl Quaternized Ammonium Salt

The monoalkyl quaternized ammonium salt cationic surfactants useful herein are those having one long alkyl chain which has about 22 carbon atoms and in one embodiment a C22 alkyl group. The remaining groups attached to nitrogen are independently selected from an alkyl group of from 1 to about 4 carbon atoms or an alkoxy, polyoxyalkylene, alkylamido, hydroxyalkyl, aryl or alkylaryl group having up to about 4 carbon atoms.

Mono-long alkyl quaternized ammonium salts useful herein are those having the formula (I):

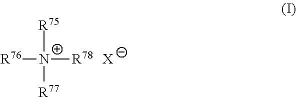

wherein one of R⁷⁵, R⁷⁶, R⁷⁷ and R⁷⁸ is selected from an alkyl group of 22 carbon atoms or an aromatic, alkoxy, polyoxyalkylene, alkylamido, hydroxyalkyl, aryl or alkylaryl group having up to about 30 carbon atoms; the remainder of R⁷⁵, R⁷⁶, R⁷⁷ and R⁷⁸ are independently selected from an alkyl group of from 1 to about 4 carbon atoms or an alkoxy, polyoxyalkylene, alkylamido, hydroxyalkyl, aryl or alkylaryl group having up to about 4 carbon atoms; and X⁻ is a salt-forming anion such as those selected from halogen, (e.g. chloride, bromide), acetate, citrate, lactate, glycolate, phosphate, nitrate, sulfonate, sulfate, alkylsulfate, and alkyl sulfonate radicals. The alkyl groups can contain, in addition to carbon and hydrogen atoms, ether and/or ester linkages, and other groups such as amino groups. The longer chain alkyl groups, e.g., those of about 22 carbons, or higher, can be saturated or unsaturated. One of R⁷⁵, R⁷⁶, R⁷⁷ and R⁷⁸ can be selected from an alkyl group of about 22 carbon atoms, the remainder of R⁷⁵, R⁷⁶, R⁷⁷ and R⁷⁸ are independently selected from CH₃, C₂H₅, C₂H₄OH, and mixtures thereof; and X is selected from the group consisting of Cl, Br, CH₃OSO₃, C₂H₅OSO₃, and mixtures thereof.

Nonlimiting examples of such mono-long alkyl quaternized ammonium salt cationic surfactants include: behenyl trimethyl ammonium salt.

### Mono-Long Alkyl Amidoamine Salt

Mono-long alkyl amines are also suitable as cationic surfactants. Primary, secondary, and tertiary fatty amines are useful. Particularly useful are tertiary amido amines having an alkyl group of about 22 carbons. Exemplary tertiary amido amines include: behenamidopropyldimethylamine, behenamidopropyldiethylamine, behenamidoethyldiethylamine, behenamidoethyldimethylamin. Useful amines in the present invention are disclosed in U.S. Pat. No. 4,275,055, Nachtigal, et al. These amines can also be used in combination with acids such as l-glutamic acid, lactic acid, hydrochloric acid, malic acid, succinic acid, acetic acid, fumaric acid, tartaric acid, citric acid, l-glutamic hydrochloride, maleic acid, and mixtures thereof; in one embodiment l-glutamic acid, lactic acid, and/or citric acid. The amines herein can be partially neutralized with any of the acids at a molar ratio of the amine to the acid of from about 1:0.3 to about 1:2, and/or from about 1:0.4 to about 1:1.

### Di-Long Alkyl Quaternized Ammonium Salt

Di-long alkyl quaternized ammonium salt can be combined with a mono-long alkyl quaternized ammonium salt or mono-long alkyl amidoamine salt. It is believed that such combination can provide easy-to rinse feel, compared to single use of a monoalkyl quaternized ammonium salt or mono-long alkyl amidoamine salt. In such combination with a mono-long alkyl quaternized ammonium salt or mono-long alkyl amidoamine salt, the di-long alkyl quaternized ammonium salts are used at a level such that the wt % of the dialkyl quaternized ammonium salt in the cationic surfactant system is in the range of from about 10% to about 50%, and/or from about 30% to about 45%.

The di-long alkyl quaternized ammonium salt cationic surfactants useful herein are those having two long alkyl chains having about 22 carbon atoms. The remaining groups attached to nitrogen are independently selected from an alkyl group of from 1 to about 4 carbon atoms or an alkoxy, polyoxyalkylene, alkylamido, hydroxyalkyl, aryl or alkylaryl group having up to about 4 carbon atoms.

Di-long alkyl quaternized ammonium salts useful herein are those having the formula (II):

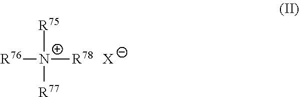

wherein two of R⁷⁵, R⁷⁶, R⁷⁷ and R⁷⁸ is selected from an alkyl group of from 22 carbon atoms or an aromatic, alkoxy, polyoxyalkylene, alkylamido, hydroxyalkyl, aryl or alkylaryl group having up to about 30 carbon atoms; the remainder of R⁷⁵, R⁷⁶, R⁷⁷ and R⁷⁸ are independently selected from an alkyl group of from 1 to about 4 carbon atoms or an alkoxy, polyoxyalkylene, alkylamido, hydroxyalkyl, aryl or alkylaryl group having up to about 4 carbon atoms; and X⁻is a salt-forming anion such as those selected from halogen, (e.g. chloride, bromide), acetate, citrate, lactate, glycolate, phosphate, nitrate, sulfonate, sulfate, alkylsulfate, and alkyl sulfonate radicals. The alkyl groups can contain, in addition to carbon and hydrogen atoms, ether and/or ester linkages, and other groups such as amino groups. The longer chain alkyl groups, e.g., those of about 22 carbons, or higher, can be saturated or unsaturated. One of R⁷⁵, R⁷⁶, R⁷⁷ and R⁷⁸ can be selected from an alkyl group of from 22 carbon atoms, the remainder of R⁷⁵, R⁷⁶, R⁷⁷ and R⁷⁸ are independently selected from CH₃, C₂H₅, C₂H₄OH, and mixtures thereof; and X is selected from the group consisting of Cl, Br, CH₃OSO₃, C₂H₅OSO₃, and mixtures thereof.

Such dialkyl quaternized ammonium salt cationic surfactants include, for example, dialkyl (C22) dimethyl ammonium chloride, ditallow alkyl dimethyl ammonium chloride, dihydrogenated tallow alkyl dimethyl ammonium chloride. Such dialkyl quaternized ammonium salt cationic surfactants also include, for example, asymmetric dialkyl quaternized ammonium salt cationic surfactants.

### High Melting Point Fatty Compound

The conditioner gel matrix of the conditioner composition includes one or more high melting point fatty compounds. The high melting point fatty compounds useful herein may have a melting point of 25° C. or higher, and is selected from the group consisting of fatty alcohols, fatty acids, fatty alcohol derivatives, fatty acid derivatives, and mixtures thereof. It is understood by the artisan that the compounds disclosed in this section of the specification can in some instances fall into more than one classification, e.g., some fatty alcohol derivatives can also be classified as fatty acid derivatives. However, a given classification is not intended to be a limitation on that particular compound, but is done so for convenience of classification and nomenclature. Further, it is understood by the artisan that, depending on the number and position of double bonds, and length and position of the branches, certain compounds having certain carbon atoms may have a melting point of less than 25° C. Such compounds of low melting point are not intended to be included in this section. Nonlimiting examples of the high melting point compounds are found in International Cosmetic Ingredient Dictionary, Fifth Edition, 1993, and CTFA Cosmetic Ingredient Handbook, Second Edition, 1992.

Among a variety of high melting point fatty compounds, fatty alcohols are suitable for use in the conditioner composition. The fatty alcohols useful herein are those having from about 14 to about 30 carbon atoms, from about 16 to about 22 carbon atoms. These fatty alcohols are saturated and can be straight or branched chain alcohols. Suitable fatty alcohols include, for example, cetyl alcohol, stearyl alcohol, behenyl alcohol, and mixtures thereof.

High melting point fatty compounds of a single compound of high purity can be used. Single compounds of pure fatty alcohols selected from the group of pure cetyl alcohol, stearyl alcohol, and behenyl alcohol can also be used. By “pure” herein, what is meant is that the compound has a purity of at least about 90%, and/or at least about 95%. These single compounds of high purity provide good rinsability from the hair when the consumer rinses off the composition.

The high melting point fatty compound can be included in the conditioner composition at a level of from about 0.1% to about 20%, alternatively from about 1% to about 15%, and alternatively from about 1.5% to about 8% by weight of the composition, in view of providing improved conditioning benefits such as slippery feel during the application to wet hair, softness and moisturized feel on dry hair.

### Aqueous Carrier

The conditioner gel matrix of the conditioner composition includes a second aqueous carrier. Accordingly, the formulations of the conditioner composition can be in the form of pourable liquids (under ambient conditions). Such compositions will therefore typically comprise a second aqueous carrier, which is present at a level of from about 20 wt % to about 95 wt %, or from about 60 wt % to about 85 wt %. The second aqueous carrier may comprise water, or a miscible mixture of water and organic solvent, and in one aspect may comprise water with minimal or no significant concentrations of organic solvent, except as otherwise incidentally incorporated into the composition as minor ingredients of other components.

The second aqueous carriers useful in the conditioner composition include water and water solutions of lower alkyl alcohols and polyhydric alcohols. The lower alkyl alcohols useful herein are monohydric alcohols having 1 to 6 carbons, in one aspect, ethanol and isopropanol. The polyhydric alcohols useful herein include propylene glycol, hexylene glycol, glycerin, and propane diol.

Leave on Composition

Rheology Modifier

In one embodiment the leave-on composition or treatment may include one or more rheology modifiers to adjust the rheological characteristics of the composition for better feel, in-use properties and the suspending stability of the composition. For example, the rheological properties are adjusted so that the composition remains uniform during its storage and transportation and it does not drip undesirably onto other areas of the body, clothing or home furnishings during its use. Any suitable rheology modifier can be used. In an embodiment, the leave-on treatment may comprise from about 0.01% to about 3% of a rheology modifier, alternatively from about 0.1% to about 1% of a rheology modifier,

The one or more rheology modifier may be selected from the group consisting of polyacrylamide thickeners, cationically modified polysaccharides, associative thickeners, and mixtures thereof. Associative thickeners include a variety of material classes such as, for example: hydrophobically modified cellulose derivatives; hydrophobically modified alkoxylated urethane polymers, such as PEG-150/decyl alcohol/SMDI copolymer, PEG-150/stearyl alcohol/SMDI copolymer, polyurethane-39; hydrophobically modified, alkali swellable emulsions, such as hydrophobically modified polypolyacrylates, hydrophobically modified polyacrylic acids, and hydrophobically modified polyacrylamides; hydrophobically modified polyethers. These materials may have a hydrophobe that can be selected from cetyl, stearyl, oleayl, and combinations thereof, and a hydrophilic portion of repeating ethylene oxide groups with repeat units from 10-300, alternatively from 30-200, and alternatively from 40-150. Examples of this class include PEG-120-methylglucose dioleate, PEG-(40 or 60) sorbitan tetraoleate, PEG-150 pentaerythrityl tetrastearate, PEG-55 propylene glycol oleate, PEG-150 distearate.

Non-limiting examples of additional rheology modifiers include acrylamide/ammonium acrylate copolymer (and) polyisobutene (and) polysorbate 20; acrylamide/sodium acryloyldimethyl taurate copolymer/isohexadecane/polysorbate 80; acrylates copolymer; acrylates/beheneth-25 methacrylate copolymer; acrylates/C10-C30 alkyl acrylate crosspolymer; acrylates/steareth-20 itaconate copolymer; ammonium polyacrylate/Isohexadecane/PEG-40 castor oil; C12-16 alkyl PEG-2 hydroxypropylhydroxyethyl ethylcellulose (HM-EHEC); carbomer; crosslinked polyvinylpyrrolidone (PVP); dibenzylidene sorbitol; hydroxyethyl ethylcellulose (EHEC); hydroxypropyl methylcellulose (HPMC); hydroxypropyl methylcellulose (HPMC); hydroxypropylcellulose (HPC); methylcellulose (MC); methylhydroxyethyl cellulose (MEHEC); PEG-150/decyl alcohol/SMDI copolymer; PEG-150/stearyl alcohol/SMDI copolymer; polyacrylamide/C13-14 isoparaffin/laureth-7; polyacrylate 13/polyisobutene/polysorbate 20; polyacrylate crosspolymer-6; polyamide-3; polyquaternium-37 (and) hydrogenated polydecene (and) trideceth-6; polyurethane-39; sodium acrylate/acryloyldimethyltaurate/dimethylacrylamide; crosspolymer (and) isohexadecane (and) polysorbate 60; sodium polyacrylate. Exemplary commercially-available rheology modifiers include ACULYN™ 28, Klucel M CS, Klucel H CS, Klucel G CS, SYLVACLEAR AF1900V, SYLVACLEAR PA1200V, Benecel E10M, Benecel K35M, Optasense RMC70, ACULYN™33, ACULYN™46, ACULYN™22, ACULYN™44, Carbopol Ultrez 20, Carbopol Ultrez 21, Carbopol Ultrez 10, Carbopol 1342, Sepigel™ 305, Simulgel™600, Sepimax Zen, and/or combinations thereof.

### Aqueous Carrier

The leave-on treatment may comprise a third aqueous carrier. Accordingly, the formulations of the leave-on treatment can be in the form of pourable liquids (under ambient conditions). Such compositions will therefore typically comprise a third aqueous carrier, which is present at a level of at least 20 wt %, from about 20 wt % to about 95 wt %, or from about 60 wt % to about 85 wt %. The third aqueous carrier may comprise water, or a miscible mixture of water and organic solvent, and in one aspect may comprise water with minimal or no significant concentrations of organic solvent, except as otherwise incidentally incorporated into the composition as minor ingredients of other components.

The third aqueous carriers useful in the leave-on treatment include water and water solutions of lower alkyl alcohols and polyhydric alcohols. The lower alkyl alcohols useful herein are monohydric alcohols having 1 to 6 carbons, in one aspect, ethanol and isopropanol. The polyhydric alcohols useful herein include propylene glycol, hexylene glycol, glycerin, and propane diol.

### Additional Components

The shampoo composition, conditioner composition and/or leave-on treatments described herein may optionally comprise one or more additional components known for use in hair care or personal care products, provided that the additional components are physically and chemically compatible with the essential components described herein, or do not otherwise unduly impair product stability, aesthetics or performance. Such additional components are most typically those described in reference books such as the CTFA Cosmetic Ingredient Handbook, Second Edition, The Cosmetic, Toiletries, and Fragrance Association, Inc. 1988, 1992. Individual concentrations of such additional components may range from about 0.001 wt % to about 10 wt % by weight of the hair care compositions.

Non-limiting examples of additional components for use in the scalp care compositions include conditioning agents, natural cationic deposition polymers, synthetic cationic deposition polymers, particles, suspending agents, paraffinic hydrocarbons, propellants, viscosity modifiers, dyes, non-volatile solvents or diluents (water-soluble and water-insoluble), pearlescent aids, foam boosters, additional surfactants or nonionic cosurfactants, pediculocides, pH adjusting agents, perfumes, dyes, bleaches, preservatives, proteins, skin active agents, sunscreens, UV absorbers, and vitamins.

1. Conditioning Agent

The hair care compositions may comprise one or more conditioning agents. Conditioning agents include materials that are used to give a particular conditioning benefit to hair. The conditioning agents useful in the hair care compositions of the present invention typically comprise a water-insoluble, water-dispersible, non-volatile, liquid that forms emulsified, liquid particles. Suitable conditioning agents for use in the hair care composition are those conditioning agents characterized generally as silicones, organic conditioning oils or combinations thereof, or those conditioning agents which otherwise form liquid, dispersed particles in the aqueous surfactant matrix.

One or more conditioning agents are present from about 0.01 wt % to about 10 wt %, from about 0.1 wt % to about 8 wt %, and from about 0.2 wt % to about 4 wt %, by weight of the composition.

### Silicone Conditioning Agent

The compositions of the present invention may contain one or more silicone conditioning agents. Examples of the silicones include dimethicones, dimethiconols, cyclic silicones, methylphenyl polysiloxane, and modified silicones with various functional groups such as amino groups, quaternary ammonium salt groups, aliphatic groups, alcohol groups, carboxylic acid groups, ether groups, epoxy groups, sugar or polysaccharide groups, fluorine-modified alkyl groups, alkoxy groups, or combinations of such groups. Such silicones may be soluble or insoluble in the aqueous (or non-aqueous) product carrier. In the case of insoluble liquid silicones, the polymer can be in an emulsified form with droplet size of about 10 nm to about 30 micrometers

### Organic Conditioning Materials

The conditioning agent of the compositions of the present invention may also comprise at least one organic conditioning material such as oil or wax, either alone or in combination with other conditioning agents, such as the silicones described above. The organic material can be nonpolymeric, oligomeric or polymeric. It may be in the form of oil or wax and may be added in the formulation neat or in a pre-emulsified form. Some non-limiting examples of organic conditioning materials include, but are not limited to: i) hydrocarbon oils; ii) polyolefins, iii) fatty esters, iv) fluorinated conditioning compounds, v) fatty alcohols, vi) alkyl glucosides and alkyl glucoside derivatives; vii) quaternary ammonium compounds; viii) polyethylene glycols and polypropylene glycols having a molecular weight of up to about 2,000,000 including those with CTFA names PEG-20 200, PEG-400, PEG-600, PEG-1000, PEG-2M, PEG-7M, PEG-14M, PEG-45M and mixtures thereof.

### EXAMPLES

### Conditioner Examples

The following examples further describe and demonstrate embodiments within the scope of the present invention. The examples are given solely for the purpose of illustration and are not to be construed as limitations of the present invention, as many variations thereof are possible without departing from the spirit and scope of the invention. Where applicable, ingredients are identified by chemical or CTFA name, or otherwise defined below.

TABLE 1
[Compositions]

| Components | Ex. 1 | Ex. 2 | Ex. 3 | Ex. 4 |
|---|---|---|---|---|
| Polyquaternium-6 *1 | 0.075 | — | | |
| Polyquaternium-6 *2 | — | — | — | — |
| Zinc pyrithione *3 | 0.75 | 0.75 | 0.75 | 0.75 |
| Sodium polynaphthalene sulfonate *4 | 0.03 | 0.03 | 0.03 | 0.03 |
| Zinc carbonate *5 | 1.6 | 1.6 | | |
| Stearamidopropyldimethylamine | 2.0 | 2.0 | 2.0 | 2.0 |
| l-glutamic acid | 0.64 | 0.64 | 0.64 | 0.64 |
| Cetyl alcohol | 2.5 | 2.5 | 2.5 | 2.5 |
| Stearyl alcohol | 4.5 | 4.5 | 4.5 | 4.5 |
| Polydimethylsiloxane *6 | 4.2 | 4.2 | 4.2 | 4.2 |
| Preservatives | 0.9 | 0.9 | 0.9 | 0.9 |
| Perfume | 0.5 | 0.5 | 0.5 | 0.5 |

| | |
|---|---|
| Deionized Water | q.s. to 100% |
| Method of preparation | I |

TABLE 2
[Compositions]

| Components | Ex. 5 | Ex. 6 | Ex. 7 | Ex. 8 | Ex. 9 | Ex. 10 | Ex. 11 |
|---|---|---|---|---|---|---|---|
| Polyquaternium-6 *1 | 0.075 | — | 0.075 | — | — | — | — |
| Polyquaternium-6 *2 | — | 0.075 | — | — | 0.075 | 0.075 | 0.050 |
| Polyquaternium-10 *7 | — | — | — | 0.075 | — | — | — |
| Zinc pyrithione *3 | 0.75 | 0.75 | 0.75 | 0.75 | 0.75 | 0.75 | 0.5 |
| Zinc carbonate *5 | 1.6 | 1.6 | 1.6 | 1.6 | 1.6 | 2.0 | — |
| Silica *8 | — | — | — | — | — | — | 1.0 |
| Behenyl trimethyl ammonium chloride | — | — | — | — | 2.5 | — | — |
| Behenyl trimethyl ammonium | 2.6 | 2.6 | 2.0 | 2.6 | — | 2.5 | 2.5 |
| methosulfate | | | | | | | |
| Dicetyl dimethyl ammonium chloride | — | — | — | — | — | 0.1 | — |
| Cetyl alcohol | 1.0 | 1.0 | 1.4 | 1.0 | 2.0 | 2.0 | 2.0 |
| Stearyl alcohol | 2.4 | 2.4 | 3.4 | 2.4 | 4.0 | 4.0 | 4.0 |
| Aminosilicone *9 | 0.5 | 0.5 | 0.5 | 0.5 | 0.5 | 0.5 | 0.5 |
| Preservatives | 0.9 | 0.9 | 0.9 | 0.9 | 0.9 | 0.9 | 0.9 |
| Perfume | 0.5 | 0.5 | 0.5 | 0.5 | 0.5 | 0.5 | 0.5 |
| Panthenol | — | — | — | — | — | 0.05 | — |
| Panthenyl ethyl ether | — | — | — | — | — | 0.03 | — |
| Deionized Water | q.s to 100% | | | | | | |
| Method of preparation | I-mod | I-mod | II | I-mod | I | I | I |
| Deposition of Zinc carbonate | S | S | S | A | — | — | — |

### Definitions of Components

*1 Polyquaternium-6: Poly(diallyldimethylammonium chloride) supplied with a tradename Merquat 100 from Nalco, having a charge density of about 6.2 meq/g, and molecular weight of about 150,000 g/mol
*2 Polyquaternium-6: Poly(diallyldimethylammonium chloride) supplied with a tradename Merquat 106 from Nalco having a charge density of about 6.2 meq/g, and molecular weight of about 15,000 g/mol
*3 Zinc pyrithione: having a particle size of from about 1 to about 10 microns
*4 Sodium polynaphthalene sulfonate having a tradename Darvan1 Spray Dried, supplied from RT Vanderbilt having a molecular weight of about 3,000 g/mol in comparison to standards of sodium poly(styrenesulfonate) and a charge density of from about 3.5 to about 4.0 meq/g
*5 Zinc carbonate: having a particle size of from about 1 to about 10 microns
*6 Polydimethylsiloxane: having a viscosity of 10,000 cSt
*7 Polyquaternium-10: Quaternized hydroxyethylcellulose supplied with a tradename Ucare Polymer JR-400 from Dow Chemical
*8 Silica: having a particle size of 0.5 to 20 microns
*9 Aminosilicone: Terminal aminosilicone which is available from GE having a viscosity of about 10,000 mPa·s, and having following formula:

(R₁)ₐG₃₋ₐ-Si-(—OSiG₂)ₙ-O—SiG₃₋ₐ(R₁)ₐ
wherein G is methyl; a is an integer of 1; n is a number from 400 to about 600; R₁ is a monovalent radical conforming to the general formula C_{q}H_{2q}L, wherein q is an integer of 3 and L is —NH₂.

### Method of Preparation

The conditioning compositions of “Ex. 1” through “Ex. 11” as shown above can be prepared by any conventional method well known in the art. They are suitably made by one of the following Methods I, I-mod, or II as shown above.

### Method I

Cationic surfactants and high melting point fatty compounds are added to water with agitation, and heated to about 80° C. The mixture is cooled down to about 55° C. and gel matrix is formed. Silicones, preservatives, zinc carbonates are added to the gel matrix with agitation. Separately, zinc pyrithione premixed in Sodium polynaphthalene sulfonate in water solution if Sodium polynaphthalene sulfonate included. Then zinc pyrithione with or without Sodium polynaphthalene sulfonate, and if included, polymers are added with agitation at about 45° C. Then, if included, other components such as perfumes are added with agitation. Then the composition is cooled down to room temperature.

### Method I-Mod

Cationic surfactants and high melting point fatty compounds are added to water with agitation, and heated to about 80° C. The mixture is cooled down to about 55° C. and gel matrix is formed. Silicones, perfumes, preservatives, zinc carbonates are added to the gel matrix with agitation. Then, zinc pyrithione, and if included, polymers are added with agitation at about 30° C. Then, if included, other components are added with agitation.

### Method II

Cationic surfactants and high melting point fatty compounds are mixed and heated to from about 66° C. to about 85° C. to form an oil phase. Separately, water is heated to from about 20° C. to about 48° C. to form an aqueous phase. In Becomix® direct injection rotor-stator homogenizer, the oil phase is injected and it takes 0.2 second or less for the oils phase to reach to a high shear field having an energy density of from 1.0×10⁵ J/m³ to 1.0×10⁷ J/m³ where the aqueous phase is already present. A gel matrix is formed at a temperature of above 50° C. to about 60° C. Silicones, preservatives, zinc carbonates are added to the gel matrix with agitation. Then, zinc pyrithione, and if included, polymers are added with agitation at about 32° C. Then, if included, other components such as perfumes are added with agitation. Then the composition is cooled down to room temperature.

### Anti-Dandruff Shampoo Examples

The following examples illustrate the present invention. The exemplified compositions can be prepared by conventional formulation and mixing techniques. It will be appreciated that other modifications of the present invention within the skill of those in the hair care formulation art can be undertaken without departing from the spirit and scope of this invention. All parts, percentages, and ratios herein are by weight unless otherwise specified. Some components may come from suppliers as dilute solutions. The levels given reflect the weight percent of the active material, unless otherwise specified.

### Anti-Dandruff Shampoo Examples

| | Ex. | Ex. | Ex. | Ex. | Ex. | Ex. | Ex. | Ex. | Ex. | Ex. |
|---|---|---|---|---|---|---|---|---|---|---|
| Ingredient | 12 | 13 | 14 | 15 | 16 | 17 | 18 | 19 | 20 | 21 |
| Water | q.s. | q.s. | q.s. | q.s. | q.s. | q.s. | q.s. | q.s. | q.s. | q.s. |
| Sodium Laureth Sulfate | 10 | 10 | 6 | 6 | — | — | — | — | — | 8 |
| (SLE3S-28% active)¹ | | | | | | | | | | |
| Sodium Laureth Sulfate | — | — | — | — | 12 | 10.5 | 11.5 | 10.5 | 16 | — |
| (SLE1S-29% active)² | | | | | | | | | | |
| Sodium Lauryl Sulfate (SLS- | 6 | 6 | 7 | 7 | — | 1.5 | — | 1.5 | — | 7 |
| 29% active)³ | | | | | | | | | | |
| Coco monoethanolamide (85% | 1.6 | 1.6 | — | — | 1.5 | 1 | 1.5 | 1 | — | — |
| active)⁴ | | | | | | | | | | |
| Cocoamdopropyl Betaine (30% | — | — | 1 | 1 | 1.5 | 1 | 1.25 | 1 | 1.7 | 2 |
| active)⁵ | | | | | | | | | | |
| Ethylene Glycol Disterate⁶ | 1.5 | 1.5 | 2.5 | 2.5 | 1.5 | 1.5 | 2.0 | 1.5 | — | 1.5 |
| Polyquaternium 76⁷ | — | — | — | — | — | 0.001 | 0.003 | 0.001 | — | — |
| Polquaternium 10⁸ | — | — | — | — | — | — | — | — | 0.075 | — |
| Polquaternium 10⁹ | | | | | 0.2 | — | — | | | |
| Polyquaternium 6¹⁰ | — | — | — | — | — | — | — | — | 0.075 | — |
| Guar, Hydroxylpropyl | 0.5 | 0.5 | 0.23 | 0.23 | 0.3 | 0.3 | 0.3 | 0.3 | — | 0.15 |
| Trimonium Chloride¹¹ | | | | | | | | | | |
| Guar, Hydroxylpropyl | — | — | — | — | — | — | — | — | 0.325 | 0.15 |
| Trimonium Chloride¹² | | | | | | | | | | |
| 330M silicone 712¹³ | 0.5 | 1.7 | 0.8 | 0.8 | 0.8 | 0.8 | 0.8 | 1.7 | — | 2.7 |
| Silicone microemulsion¹⁴ | — | — | — | — | — | — | — | — | 0.75 | — |
| Trihydroxysteam¹⁵ | — | — | — | — | — | — | — | — | 0.1 | — |
| Zinc Pyrithone¹⁶ | 1.0 | 1.0 | 1.0 | 1.0 | 1.0 | 1.0 | 1.0 | 2 | 1 | 1 |
| Zinc Carbonate¹⁷ | 1.6 | 1.6 | 1.6 | 1.6 | 1.6 | 1.6 | 1.6 | 1.6 | 1.6 | — |
| Gel Network¹⁸ | — | — | — | — | — | — | — | — | 17.7 | — |
| Magnesium Sulfate | 0.28 | 0.28 | | | | | | | | |
| Cetyl Alcohol | 0.6 | 0.6 | | | | | | | | |
| Menthol¹⁹ | — | 0.5 | 0.5 | 0.5 | 0.5 | 0.5 | 0.5 | 0.5 | — | — |
| Fragrance | 0.7 | 0.7 | 0.6 | 0.6 | 0.7 | 0.7 | 0.7 | 0.7 | 0.7 | 0.7 |

| | |
|---|---|
| Sodium Chloride, Sodium | Adjust as needed for viscosity of about 5,000-15,000 cps |
| Xylene Sulfonate²⁰ | at 2/s shear rate |
| Preservatives, pH adjusters | Up to 1% |

¹Sodium Laureth (3 molar ethylene oxide) Sulfate at 28% active, supplier: P&G
²Sodium Laureth (1 molar ethylene oxide) sulfate at 29% active, supplier: P&G
³Sodium Lauryl Sulfate at 29% active, supplier: P&G
⁴Coco monethanolamide at 85% active, supplier: Stephan Co or supplier Evonik
⁵Tegobetaine F-B, 30% active, supplier: Goldschmidt Chemical
⁶Ethylene Glycol Disterate at 100% active, supplier: Goldschmidt Chemical or supplier Evonik
⁷Acrylamide: Triquat cationic polymer, 10% solution, trade name: Mirapol AT from Rhodia,
⁸KG30M cationic cellulose polymer from Amerchol/Dow
⁹JR30M cationic cellulose polymer from Amerchol/Dow
¹⁰PolyDADMAC at 31.5% active, trade name: Mirapol 100S from Rhodia
¹¹Cationic Guar with M.Wt. of ~500,000 and a Charge Density of 0.8 meq/g - Solvay/Rhodia
¹²Cationic Guar with M.Wt. of ~1,300,000 and a Charge Density of 0.8 meq/g - ASI
¹³330M silicone, 100% active, supplier: Momentive (silicone used by P&G to make a 70% active, 30 um emulsion)
¹⁴Belsil 3560 VP silicone microemulsion from Wacker, 60,000 cst internal viscosity of silicone, approx. 125 nm
¹⁵Thixin R from Rheox Inc.
¹⁶ZPT from Arch Chemical
¹⁷Zinc carbonate from the Bruggeman Group
¹⁸Gel Networks; See Composition below. The water is heated to about 74° C. and the Cetyl Alcohol, Stearyl Alcohol, and the SLES Surfactant are added to it. After incorporation, this mixture is passed through a heat exchanger where it is cooled to about 35° C. As a result of this cooling step, the Fatty Alcohols and surfactant crystallized to form a crystalline gel network.
¹⁹Menthol from Symrise
²⁰Sodium Chloride USP (food grade), Sodium Xylene Sulfonate 30% active

### Scalp Leave-on Treatment (LOT) Examples

In an embodiment of the present invention, the following are non-limiting examples: The exemplified compositions can be prepared by conventional formulation and mixing techniques. It will be appreciated that other modifications of the present invention within the skill of those in the hair care formulation art can be undertaken without departing from the spirit and scope of this invention. All parts, percentages, and ratios herein are by weight unless otherwise specified. Some components may come from suppliers as dilute solutions. The levels given reflect the weight percent of the active material, unless otherwise specified

| | 22 | | | | |
|---|---|---|---|---|---|
| | Active | 23 | 24 | 25 | 26 |
| | wt | Active | Active | Active | Active |
| | %) | wt % | wt % | wt % | wt % |
| Water | Q.S. | QS | QS | QS | QS |
| Alcohol 100% | 50 | 50 | 0 | 50 | 60 |
| (Ethanol) | | | | | |
| Isoproryl Alcohol | 0 | 0 | 0 | 0 | 0 |
| Acrylates/C10-30 | 0.35 | 0.5 | 0.2 | 0 | 0 |
| alkyl acrylate | | | | | |
| crosspolymer *1 | | | | | |
| Carbomer *2 | 0 | 0 | 0 | 0 | 0 |
| Polyacrylamide *3 | 0 | 0 | 0 | 0.5 | 0 |
| C13-14 Isoparaffin *3 | 0 | 0 | 0 | 0.5 | 0 |
| Laureth 7 *3 | 0 | 0 | 0 | 0.1 | 0 |
| Polyacrylate | 0 | 0 | 0 | 0 | 0.5 |
| crosspolymer-6 *4 | | | | | |
| Dehydroxanthan | 0 | 0 | 0 | 0.25 | 0 |
| Gum *5 | | | | | |
| Cetyl Alcohol, | 0 | 0 | 0 | 0 | 0 |
| Sodium | | | | | |
| Polyacrylate, | | | | | |
| Glyceryl Stearate, | | | | | |
| Polysorbate 80, and | | | | | |
| Caprylic/Capric | | | | | |
| Triglycerinde *6 | | | | | |
| Acrylates/ | 0 | 0 | 0 | 0 | 0 |
| Aminoacrylates/ | | | | | |
| C10-30 Alkyl | | | | | |
| PEG-20 Itaconate | | | | | |
| Copolymer *7 | | | | | |
| Zinc pyrithione *8a | 0.1 | 0.2 | 0.07 | 0.1 | 0.1 |
| Zinc carbonate *8b | 0 | 0.2 | 0 | 0 | 0 |
| PEG/PPG 20/23 | 0 | 1 | 0 | 0 | 0 |
| Dimethicone 430 *9 | | | | | |
| Bis-PEG/PPG- | 0.7 | 0 | 0 | 1 | 0 |
| 16/16 PEG/PPG | | | | | |
| 16/16 | | | | | |
| Dimethicone *10 | | | | | |
| Polyquaternium-4 | 0 | 0 | 0 | 0 | 0 |
| *111 | | | | | |
| Panthenol | 0.15 | 0.5 | 0 | 0.15 | 0 |
| Niacinamide | 2.5 | 0 | 0 | 3 | 0 |
| Caffeine | 0.75 | 0 | 0 | 1.25 | 0 |
| Glycerin | 0.5 | 5 | 0 | 5 | 0 |
| Argania Spinosa | 0 | 0 | 0 | 0 | 0 |
| Kernel Oil *12 | | | | | |
| Propylene Glycol | 0 | 0 | 1 | 0 | 0 |
| Menthol | 0 | 0 | 0.5 | 0 | 0 |
| Polyvinylpyrrolidone *13 | 0 | 1 | 0 | 0 | 0 |
| Polyethylene Low | 0 | 0.5 | 0 | 0 | 0 |
| Density Powder *14 | | | | | |
| Tapicoa Starch | 0 | 0 | 1 | 0 | 0 |
| Polymethylsilsesuioxane | | | | | |
| *15 | | | | | |
| Benzyl Alcohol | 0 | 0 | 0.5 | 0 | 0 |
| Methylisothiazolinone | 0 | 0 | 0.05 | 0 | 0 |
| *16 | | | | | |
| PEG-40 | 0 | 0 | 0.5 | 0 | 0 |
| Hydrogenated Castor | | | | | |
| Oil *17 | | | | | |
| Tetrahydroxypropyl | 0.12 | 0 | 0.14 | 0 | 0 |
| Ethylenediamine *18 | | | | | |
| Triethanolamine *19 | 0 | 0.1 | 0 | 0 | 0 |
| Glycolic Acid *20 | 0 | 0 | 0 | 0 | 0 |
| Citric Acid | 0 | 0 | 0 | 0.008 | 0.005 |

*1 as in Carbopol Ultrez 21 available from Lubrizol
*2 as in Carbopol Ultrez 30 available from Lubrizol
*3 as in Sepigel 305 from Seppic
*4 as in SepiMax Zen from Seppic
*5 as in Amaze XT from AkzoNovel
*6 as in Jeesperse CPW-CG-02 from Jeen
*7 as in Structure Plus from Akzo Nobel
*8a as in ZPT from Lonza Personal Care
*8b as in Zinc carbonate from Brueggemann Chemical
*9 as in Silsoft 430 Dimethicone Copolyol from Momentive
*10 as in Abil Care 85 from Evonik
*11 as in Celquat H-100 from Akzo Nobel
*12 as in Lipofructyl Argan LS9779 from BASF
*13 as in PVP K-30 from ISP Technologies
*14 as in Microthene FN 510-00 from Equistar Chemicals
*15 as in Dry Flo TS from Akzo Nobel
*16 as in Neolone 950 from Rohm and Haas
*17 as in Cremophor RH-40 Surfactant from BASF
*18 as in Neutrol Te from BASF
*19 as in Trolamine from Dow Chemical
*20 as in Glypure from DuPont

| | Examples 27 |
|---|---|
| | Active wt %) |
| Water | Q.S. |
| Alcohol 100% | 50 |
| (Ethanol) | |
| Acrylates/C10-30 | 0.35 |
| alkyl acrylate | |
| crosspolymer *1 | |
| Bis-PEG/PPG- | 0.7 |
| 16/16 PEG/PPG | |
| 16/16 | |
| Dimethicone *2 | |
| Panthenol *3 | 0.15 |
| Niacinamide *4 | 2.5 |
| Caffeine *5 | 0.75 |
| Climbazole *6 | 0.50 |
| Butylene Glycol *7 | 1.0 |
| Benzyl Alcohol *8 | 0.5 |
| Tetrahydroxypropyl | 0.05 |
| Ethylenediamine *9 | |

*1 as in Carbopol Ultrez 21 available from Lubrizol
*2 as in Abil Care 85 from Evonik
*3 as in Dexapaanthenol USP from Roche vitamins
*4 as in Niancinamide USP from Roche Vitamins
*5 as in Caffeine USP from BASF
*6 as in Crinipan AD from Symrise
*7 as in Cosmetic Quality 1,3-Butylene Glycol from Oxea Corporation
*8 as in Benzyl Alcohol from Polarome International
*9 as in Neutrol Te from BASF

### Example

### Clinical Evaluation of Scalp and Hair Health

Measures and Method:

The measures of scalp and hair health are related to quantification of the degree of oxidative stress. Exemplified herein is the quantitation of (±)-9-hydroxy-10E, 12Z-octadecadienoic acid and (±)-13-hydroxy-10E, 12Z-octadecadienoic acid (“HODE”). Quantitation of oxidized lipids from extracts of the adhesive article, tape strips, can be conducted using gradient reversed-phase high performance liquid chromatography with tandem mass spectrometry (HPLC/MS/MS).

Tape strips (single or multiple tape strips) obtained from the scalp of human subjects are placed into individual polypropylene amber vials or glass amber vials, and then extracted with extraction solvent (methanol with 0.1% butylated hydroxytoluene, w/v) using vortexing for 10 min. Hair samples from the head are simultaneously pulverized and extracted using the extraction solvent in a bead-based device. The standards and the extracts of the scalp tape strips and hair extracts are analyzed using gradient reversed-phase high performance liquid chromatography with tandem mass spectrometry (HPLC/MS/MS). Analytes (oxidized or non-oxidized lipids) and the ISTDs are monitored by positive ion electrospray (ESI). A standard curve is constructed by plotting the signal, defined here as the peak area ratio (peak area analyte/peak area ISTD) or peak area analyte only, for each standard versus the mass of each analyte for the corresponding standard. The mass of each analyte in the calibration standards and human scalp extract samples are then back-calculated using the generated regression equation. The result can be reported as the mass of oxidized lipid/tape strip or the result can be standardized by dividing by the amount of oxidized lipid by the amount of the corresponding parent non-oxidized lipid that is also found in the tape strip extract. Additionally results could be reported by standardizing the amount of oxidized lipid by the amount of corresponding protein found in the tape strip extract. Standardization could also be done by collecting the cells removed, drying them and weighing them.

Clinical Protocol.

The general clinical protocol involves recruitment of a compromised scalp health population (represented by the presence of dandruff or seborrheic dermatitis) and a comparative healthy scalp group. The differentiation of healthy and unhealthy populations is based on expert assessment of severity of flaking as measured by the Adherent Scalp Flaking Score (ASFS) and described in detail in Bacon, R., H. Mizoguchi, and J. Schwartz, Assessing therapeutic effectiveness of scalp treatments for dandruff and seborrheic dermatitis, part 1: a reliable and relevant method based on the adherent scalp flaking score (ASFS). J Derm Treat, 2014. 25: p. 232-236. The unhealthy scalp population needs to meet a study entrance criterion of ASFS≧24 whereas the healthy scalp population is ASFS≦8.

All potential clinical subjects begin a pre-clinical wash-out period of 8 weeks utilizing a standard cosmetic shampoo, which is a conventional shampoo with no scalp active material. The length of time is designed to assure that hair sampled at baseline had sufficient time to grow under known scalp conditions. Including 8 weeks of pre-treatment, this is a 24 week, multi-site, double-blind randomized study. Participants are healthy male and female subjects 18-75 years old. Subjects are non-smokers for 5 or more years, have not chemically treated their hair in two months prior to the start of washout, agree not to cut their hair within sampling and measurement site and agree not to chemically treat hair for the duration of the study. Subjects do not have hair shorter than two inches during the study or be balding. Subjects have not have been pregnant or lactating for the past three months or planning to become pregnant during the study. Subjects do not have skin diseases of the scalp such as psoriasis, psoriasiform eczema, lichenoid eruption, tinea capitis or other scalp infections or infestations.

At baseline of the study, approximately 60 healthy scalp and 308 unhealthy scalp individuals are enrolled in the study based on ASFS quantitation. Their hair is sampled by trimming close to the scalp as well as tape strips acquired for subsequent evaluation of oxidative stress status. The unhealthy scalp individuals are randomly placed on a treatment protocol which involved using either a cosmetic shampoo (the same as in pre-treatment phase), a zinc pyrithione-based scalp care shampoo (approx. 120 per treatment leg) or a regimen of cosmetic shampoo and a scalp care leave-on treatment (LOT) comprising zinc pyrithione (approx. 68). Shampoos are utilized at least three times per week with a refrain of 72 hours prior to any subsequent assessments of flaking severity. The LOT is used seven times per week, with a refrain of 24 hours prior to assessments. Oxidative stress measurements on scalp are reported at Weeks 0 (baseline) 3 and 16. Due to the time required for new hair to be grown (approximately 1 cm/month) and the assurance of the nature of the scalp condition during growth, hair is sampled only at baseline and Week 16. For the purposes of assessment of oxidative stress in the hair samples, only the bottom (most recent) 2 cm of growth is used and all hair is cleaned thoroughly prior to evaluation.

Results:

HODE is used as a representative biomarker of the level of oxidative stress being experienced by either the scalp or hair (Spiteller, P. and G. Spiteller 9-Hydroxy-10,12-octadecadienoic acid (9-HODE) and 13-hydroxy-9,11-octadecadienoic acid (13-HODE): excellent markers for lipid peroxidation Chem Phys Lipids 1997, 89, 131-9. In all cases, the level of HODE is expressed as a ratio (normalized) to separately measured levels of parent linoleic acid from the same sample. Results are expressed as either the logarithm of the absolute level or, for treatment data, the percent reduction in the ratio as compared to baseline.

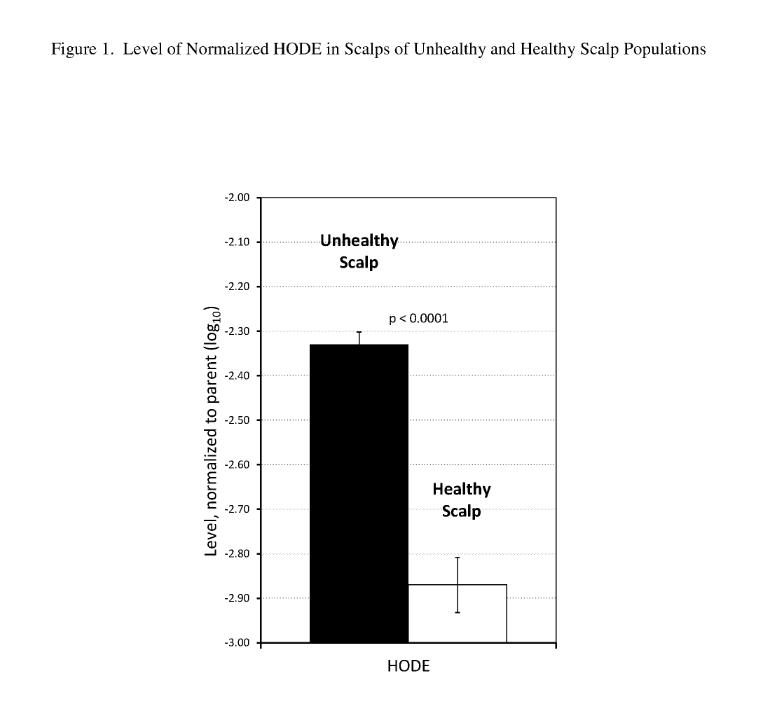

In comparing the level of normalized HODE found in the scalps of healthy and unhealthy scalp populations, FIG. 1 demonstrates that the unhealthy scalp population has significantly higher levels of normalized HODE in the scalp than the comparative healthy scalp population:

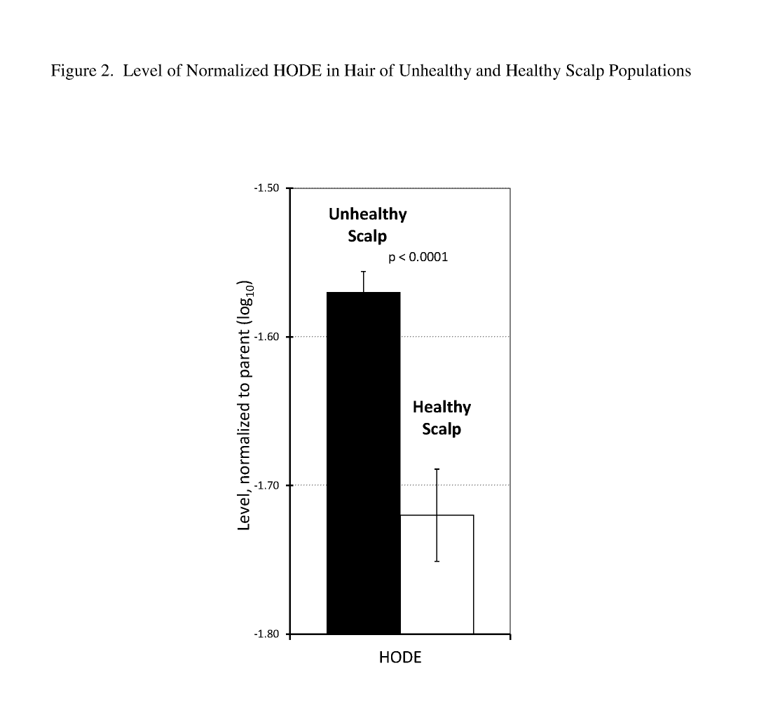

In comparing the level of normalized HODE found in the hair of healthy and unhealthy scalp populations, FIG. 2 demonstrates that the unhealthy scalp population has significantly higher levels of normalized HODE in the hair than the comparative healthy scalp population:

These observations on healthy and unhealthy scalp populations validate HODE as a relevant biomarker differentiating the two conditions. Further, since these effects are observed in both scalp and hair samples, it establishes the correlation between scalp health and resultant hair health.

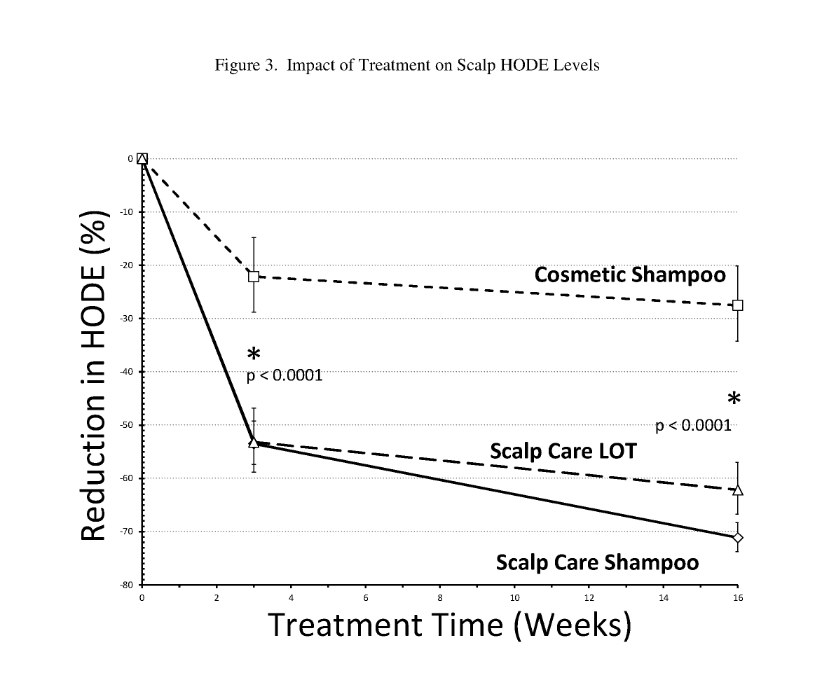

Treatment of the unhealthy sub-population with either cosmetic or scalp care shampoo or a cosmetic shampoo/scalp care LOT further demonstrates the correlation between scalp and hair condition and the method of improving hair by first improving the scalp condition. Quantitation of HODE level in scalp as a function of treatment type is summarized in FIG. 3:

The data demonstrates the effective reduction in HODE levels by a product designed to improve scalp health.

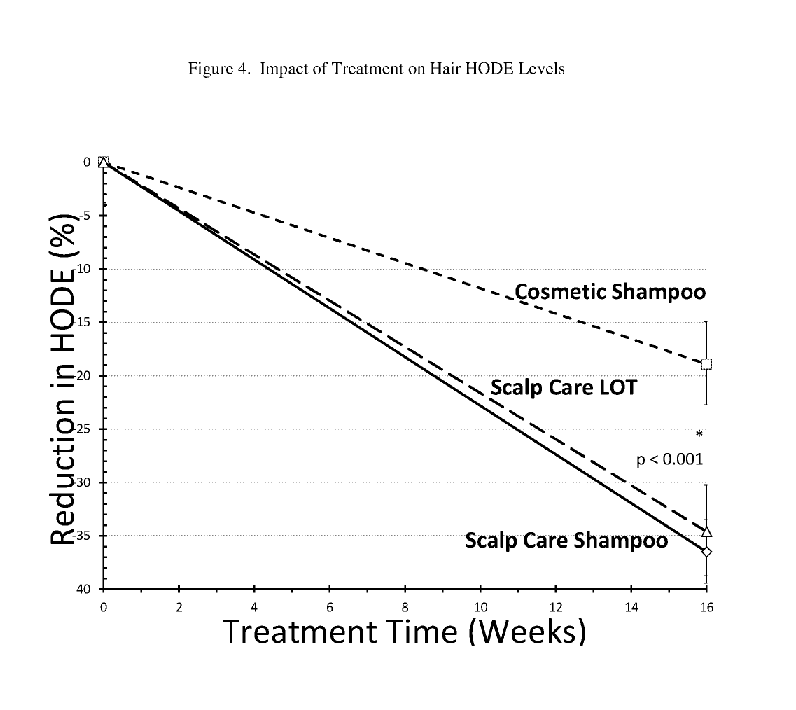

Quantitation of HODE level in hair as a function of treatment type is summarized in FIG. 4:

The concomitant reduction in hair HODE levels as a function of treatment with either a scalp health improvement shampoo or a scalp care LOT demonstrates the benefits to the hair quality and health that result from improving scalp health.

In an embodiment of the present invention, there is at least a 10% reduction in a oxidative stress biomarker in pre-emergent hair as demonstrated from emergent hair following application with an scalp care composition, when compared to a non-scalp care composition, In a further embodiment, there is at least a 15% reduction in a oxidative stress biomarker in pre-emergent hair as demonstrated from emergent hair following application with an scalp care composition, when compared to a non-scalp care composition. In a further embodiment, there is at least a 20% reduction in an oxidative stress biomarker in pre-emergent hair as demonstrated from emergent hair following application with a scalp care composition, when compared to a non-scalp care composition. In yet a further embodiment, there is at least a 25% reduction in a oxidative stress biomarker in pre-emergent hair as demonstrated from emergent hair following application with an scalp care composition, when compared to a non-scalp care composition.

In the examples, all concentrations are listed as weight percent, unless otherwise specified and may exclude minor materials such as diluents, filler, and so forth. The listed formulations, therefore, comprise the listed components and any minor materials associated with such components. As is apparent to one of ordinary skill in the art, the selection of these minors will vary depending on the physical and chemical characteristics of the particular ingredients selected to make the hair care composition.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm.”

Every document cited herein, including any cross referenced or related patent or application and any patent application or patent to which this application claims priority or benefit thereof, is hereby incorporated herein by reference in its entirety unless expressly excluded or otherwise limited. The citation of any document is not an admission that it is prior art with respect to any invention disclosed or claimed herein or that it alone, or in any combination with any other reference or references, teaches, suggests or discloses any such invention. Further, to the extent that any meaning or definition of a term in this document conflicts with any meaning or definition of the same term in a document incorporated by reference, the meaning or definition assigned to that term in this document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. A method of improving the health of hair emerging from a scalp comprising a reduction of oxidative stress in the scalp by application of a composition resulting in reduction in oxidative stress in pre-emergent hair as demonstrated by reduced oxidative stress in emergent hair.

2. A method according to claim 1 wherein oxidative stress is measured by a level of a biomarker.

3. A method according to claim 2 wherein one or more biomarkers is selected from the group consisting of Myeloperoxidase, (±)-9-hydroxy-10E, 12Z-octadecadienoic acid and (±)-13-hydroxy-10E, 12Z-octadecadienoic acid (HODE), squalene hydroperoxide, heat shock protein 27 (HSP27), oxidative modification of proteins, DNA oxidation and hydroxylated nucleotides, isoprostanes, α,β-unsaturated aldehydes, reaction products of α,β-unsaturated alkenals with protein and mercapturic acid pathway, early glycation adducts (EGA) and advanced glycation end products (AGE), antioxidants as biomarkers of oxidative stress and mixtures thereof.

4. A method according to claim 2 wherein there is a change in level of biomarker when compared to a baseline level of biomarker.

5. A method according to claim 2 wherein there is a change in biomarker level following application with a composition, when compared to a baseline level of biomarker prior to the application with a composition.

6. A method according to claim 5 wherein there is a reduction in an oxidative stress biomarker in pre-emergent hair as demonstrated from emergent hair following application with a scalp care composition, when compared to a non-scalp care composition.

7. A method according to claim 6 wherein there is at least a 10% reduction in oxidative stress biomarker.

8. A method according to claim 7 wherein there is at least a 10% reduction in oxidative stress biomarker following application with a scalp care composition comprising zinc pyrithione.

9. A method according to claim 7 wherein the oxidative stress biomarker is (±)-9-hydroxy-10E, 12Z-octadecadienoic acid and (±)-13-hydroxy-10E, 12Z-octadecadienoic acid (HODE).

10. A method according to claim 1 wherein the composition comprises a scalp care composition.

11. A method according to claim 10 wherein the scalp care composition comprises a scalp active material.

12. A method according to claim 11 wherein the scalp active material is selected from the group consisting of anti-dandruff actives, anti-microbial actives, anti-fungal actives and mixtures thereof.

13. A method according to claim 12 wherein the scalp active material is selected from pyridinethione salts, azoles, selenium sulphide, particulate sulfur, keratolytic agents and mixtures thereof.

14. A method according to claim 13 wherein the scalp active material is selected from zinc pyrithione, climbasole, octopirox and mixtures thereof.

15. A method according to claim 14 wherein the scalp active material is zinc pyrithione.
